(19) United States
(12) Patent Application Publication
(10) Pub. No.: US 2016/0376247 A1
(43) Pub. Date: Dec. 29, 2016

(54) NOVEL TEMPERATURE-SENSITIVE FLUORESCENT COMPOUND AND APPLICATION THEREOF

(71) Applicant: SHANGHAI INSTITUTES FOR BIOLOGICAL SCIENCES, CHINESE ACADEMY OF SCIENCES, Shanghai (CN)

(72) Inventors: Jiansheng Kang, Shanghai (CN); Taorong Xie, Shanghai (CN); Chunfeng Liu, Shanghai (CN)

(73) Assignee: SHANGHAI INSTITUTES FOR BIOLOGICAL SCIENCES, CHINESE ACADEMY OF SCIENCES, Shanghai (CN)

(21) Appl. No.: 15/109,100

(22) PCT Filed: Dec. 31, 2014
(86) PCT No.: PCT/CN2014/096005

§ 371 Date: Jun. 29, 2016

(30) Foreign Application Priority Data
Dec. 31, 2013 (CN) 201310754132.9

(51) Int. Cl. C07D 311/82; G01K 11/20; C09K 11/06; C07D 471/22; C07D 471/04

(52) U.S. Cl. CPC C07D 311/82; C07D 471/22; C07D 471/04; C09K 11/06; G01K 11/20; C09K 2211/1088; C09K 2211/1048; C09K 2211/1044

(57) ABSTRACT

Provided is a compound represented by formula I, wherein R₉ is a C1-C22 hydrocarbyl group, or a C2-C3 ester-substituted C1-C3 alkyl group, R₅, R₆, R₇, and R₈ are alkyl groups or H, and R₁, R₂, R₃, and R₄ are H or a lower hydrocarbyl group. Or, R₉ is a C2-C22 hydrocarbyl group, or a C2-C3 ester-substituted C1-C3 alkyl group, and R₅ is connected to R₁, R₆ is connected to R₂, R₇ is connected to R₃, R₈ is connected to R₄ to form six-membered rings. The present compound is temperature-sensitive and can enter into cells. An intracellular temperature distribution image having high spatial and temporal resolution can thus be obtained. The present compound can also perform distribution calibration on a temperature-sensitive fluorescent compound. Also provided is a method for measuring temperature distribution within a living cell, and a corresponding detection kit. The method satisfies the requirements of small size measurement and rapid measurement, thereby achieving high resolution in terms of space and time.

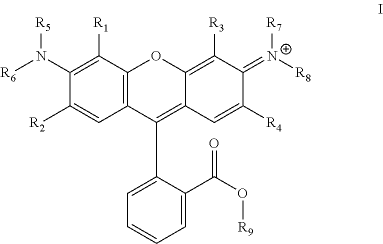

### TECHNICAL FIELD

The present invention relates to the field of cell-detection. In particular, the present invention relates to novel fluorescent dyes and the use of such novel fluorescent dyes to detect the temperature distribution within living cells.

### BACKGROUND

For cellular activities, such as metabolism, enzymatic reactions, cell division, gene expression and the like, temperature of the cell will change to some degree. These cellular activities are generally accompanied by the release of chemical energy of ATP, and heat will be generated so that the temperature will rise. In addition, under external stimuli of drugs or signals, the metabolic activity of a cell will rapidly change, leading to vigorous fluctuate of intracellular temperature. However, due to the heat exchange from extracellular environment, the changes in intracellular temperature generally are relatively local, and exhibite transient characteristics, therefore, it is difficult to measure the changes in intracellular temperature by using conventional methods of temperature measurement.

It was reported that infrared thermography methods were used to study the heat-generating effects of UCP2 living cells. The principle of infrared thermography is based on the fact that all of the objects will emit a certain amount of temperature-relevant blackbody radiation, that is, the temperature of cells can not be seperated from the temperature of their environment (medium) by using infrared thermography methods. In addition, the operating wavelength of an infrared camera is generally 14 μm, and according to Rayleigh criterion on optical resolution, the camera working in such infrared wavelength can not distinguish individual cells. Therefore, the infrared thermography method is not suitable for detecting intracellular temperature. Thermocouple is often used as a probe of a temperature-measuring device to measure the temperature change of an object, and the scanning thermography microscope was developed by replacing the probe of a scanning tunneling microscope or atomic force microscope with a thermocouple. Since the probe of thermocouple is relative rigid, such method is generally only used in the electronics industry, so as to obtain a two-dimensional micron or nano-sized thermography. In recent reports, some scholars have designed a novel thermocouple material for measuring the real-time temperature of individual cells, and the method can obtain temperature curve with high temporal resolution, but this is only a single point measurement, and for obtaining two-dimensional thermography, time resolution is significantly decreased, and such contact-type measurement will likely penetrate and damage the cell membrane. Therefore, thermal imaging of a cell can not easily be performed by a thermocouple-based temperature measurement. In recent years, some scholars have reported that temperature-sensitive fluorescent nanomaterials can be used in the detection of changes in cell temperature [1], wherein changes in mean fluorescence intensity can represent changes in the average temperature of the whole cell before and after drug stimulation. However, it is necessary to introduce such temperature-sensitive fluorescent nano-materials into cells by injection, resulting in interference and damage to cells; and it can be seen from the reported fluorescence images that distribution of the nanomaterial on the cell is very uneven, and only some small bright spots can be found [1]. In addition to the temperature, the fluorescence intensity of temperature-sensitive fluorescent materials is also relevant to the concentration distribution, therefore, there may be certain problems that the temperature of a cell is simply indicated by the average fluorescence intensity of the whole cell.

In summary, it is an urgent need in the art to develop new fluorescent dyes, so that it can meet the requirements on small size and rapid measurement for measuring intracellular temperature, achieving high spatial and temporal-resolution, and obtaining an intracellular temperature distribution image with high spatial and temporal-resolution.

### SUMMARY OF THE INVENTION

The purpose of the present invention is to provide a temperature-sensitive fluorescent dye wich can be located at cell membrane or permeable to the cell membrane and enter the cell, thereby performing accurate, easy and quick measurement of intracellular temperature.

The purpose of the present invention is also to provide a calibration method for the distribution of a temperature-sensitive fluorescent compound when using the temperature-sensitive fluorescent compound to measure the temperature distribution inside a living cell, and the compound used in the calibration method.

In a first aspect, a compound of formula I is provided in the present invention,

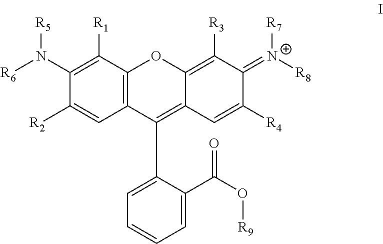

wherein,

R₉ is a hydrocarbyl of 1-22 carbon atoms, or an alkyl of 1 to 3 carbon atoms substituted with an ester group of 2-3 carbon atoms,

R₅, R₆, R₇, R₈ are a hydrocarbyl or H, and

R₁, R₂, R₃, R₄ are H or a lower hydrocarbyl; or

R₉ is a hydrocarbyl of 2-22 carbon atoms, or an alkyl of 1 to 3 carbon atoms substituted with an ester group of 2-3 carbon atoms, and

R₅ and R₁, R₆ and R₂, R₇ and R₃, R₈ and R₄ are connected into a six-membered ring.

In a preferred embodiment, the hydrocarbyl can be an alkyl, alkenyl or alkynyl; preferably, a straight-chain or branched-chain or cyclic alkyl, e.g., methyl, ethyl, propyl, isopropyl, butyl, t-butyl, pentyl, cyclopentyl, cyclohexyl and the like; preferably, a straight-chain alkyl, such as methyl, ethyl, propyl, butyl, pentyl, and the like; more preferably, methyl or cetyl.

In a preferred embodiment, R₅, R₆, R₇, R₈ is an alkyl, alkenyl or alkynyl; in a further preferred embodiment, R₅, R₆, R₇, R₈ is a lower alkoxy; preferably, R₅, R₆, R₇, R₈ is an alkyl of 1 to 8 carbon atoms; more preferably, R₅, R₆, R₇, R₈ is an alkyl of 1 to 3 carbon atoms; and most preferably, R₅, R₆, R₇, R₈ is ethyl.

In a preferred embodiment, the alkyl of 1 to 3 carbon atoms substituted with an ester group can be a methyl, ethyl or propyl, preferably methyl; and the ester group of 2-3 carbon atoms can be an ethyl ester group, propyl ester group.

In a preferred embodiment, the lower hydrocarbyl is an alkyl, alkenyl or alkynyl of 1-8 carbon atoms; preferably, an alkyl of 1-3 carbon atoms; more preferably, methyl, ethyl or propyl.

In a particular embodiment, the compound is a compound of following formula:

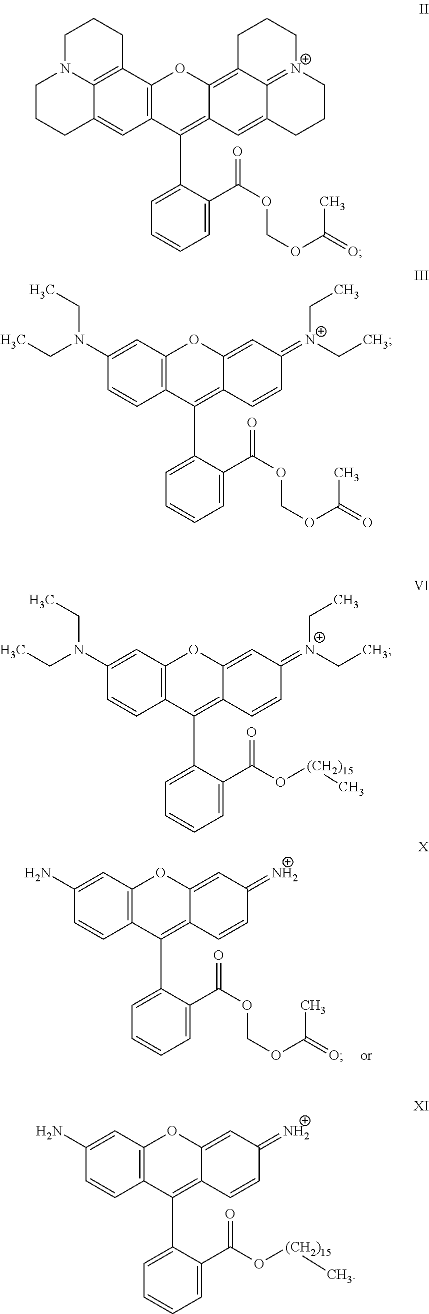

In a second aspect, use of the compound of formula I in the measurement of temperature distribution in a living cell is provided in the present invention,

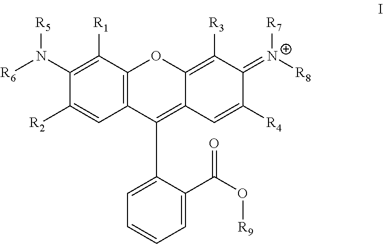

wherein,

R₉ is selected from the group consisting of a hydrocarbyl of 1-22 carbon atoms, or an alkyl of 1 to 3 carbon atoms substituted with an ester group of 2-3 carbon atoms, and an alkyl of 1 to 3 carbon atoms substituted with an aryl;

each of R₅, R₆, R₇, and R₈ is independently selected from a hydrocarbyl, and R₁, R₂, R₃, R₄ are H or a lower hydrocarbyl; or

R₉ is selected from the group consisting of a hydrocarbyl of 1-22 carbon atoms, or an alkyl of 1 to 3 carbon atoms substituted with an ester group of 2-3 carbon atoms, and an alkyl of 1 to 3 carbon atoms substituted with an aryl; and

R₅ and R₁, R₆ and R₂, R₇ and R₃, R₈ and R₄ are connected into a six-membered ring. In a preferred embodiment, each of R₅, R₆, R₇, and R₈ is independently selected from an alkyl, alkenyl or alkynyl; in a further preferred embodiment, each of R₅, R₆, R₇, and R₈ is independently selected from a lower alkyl; preferably, each of R₅, R₆, R₇, and R₈ is independently selected from an alkyl of 1 to 8 carbon atoms; more preferably, each of R₅, R₆, R₇, and R₈ is independently selected from an alkyl of 1 to 3 carbon atoms; more preferably, each of R₅, R₆, R₇, and R₈ is independently selected from methyl or ethyl; more preferably, all of R₅, R₆, R₇, and R₈ are methyl or ethyl; and most preferably, each of R₅, R₆, R₇, R₈ is ethyl.

In a preferred embodiment, the lower hydrocarbyl is an alkyl, alkenyl or alkynyl of 1-8 carbon atoms; preferably, an alkyl of 1-3 carbon atoms; more preferably, methyl, ethyl or propyl.

In a preferred embodiment, a hydrocarbyl of 1-22 carbon atoms can be an alkyl, alkenyl or alkynyl of 1 to 22 carbon atoms; preferably, a straight or branched chain or cyclic alkyl of 1-22 carbon atoms, e.g., methyl, ethyl, propyl, isopropyl, butyl, t-butyl, pentyl, cyclopentyl, cyclohexyl and the like; preferably, a straight-chain alkyl, such as methyl, ethyl, propyl, butyl, pentyl, and the like; and more preferably, methyl or hexadecyl; the alkyl of 1-3 carbon atoms can be methyl, ethyl or propyl, preferably methyl; the ester group of 2-3 carbon atoms can be an ethyl ester group, propyl ester group, preferably ethyl ester group; the alkyl of 1-3 carbon atoms substituted by an aryl is a methyl substituted by an aryl, ethyl substituted by an aryl or propyl substituted by an aryl; preferably a methyl substituted by an aryl; and more preferably an methyl substituted by a substituent of formula IX

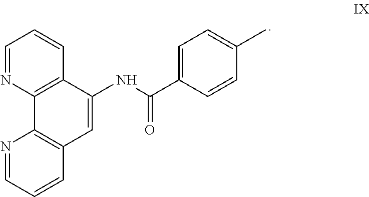 [SMILES: Cc1ccc(C(=O)Nc2cc3cccnc3c3ncccc23)cc1]

In a specific embodiment, the compound is a compound of the following formula:

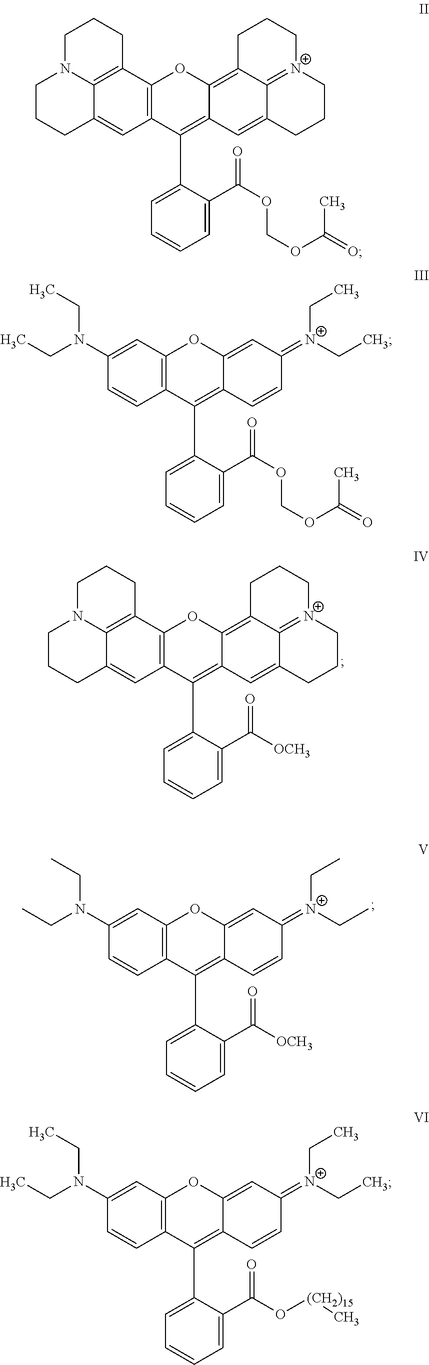

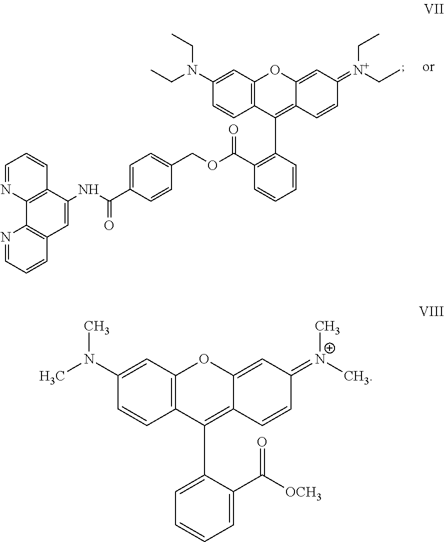

In another specific embodiment, the temperature distribution within a living cell is the temperature distribution in a subcellular structure; preferably, the subcellular structure is cell membrane, cytoplasm, or mitochondria.

In a preferred embodiment, the use is to use the compound of formula II or formula III for measuring the temperature distribution of cytoplasm within a living cell.

In another preferred embodiment, the use is to use the compound of formula IV or formula V for measuring the temperature distribution of mitochondria within a living cell.

In another preferred embodiment, the use is to use the compound of formula VI for measuring the temperature distribution of cell membrane of a living cell.

In another preferred embodiment, the use is to use the compound of formula VII, VIII for measuring the temperature of mitochondria within a living cell.

In a preferred embodiment, the compound of Formula II or Formula IV is used in the measurement of temperature through anti-Stokes luminescence imaging.

In another preferred embodiment, the compound of Formula III, Formula V, Formula VI, Formula VII or Formula VIII is used in the measurement of temperature through Stokes luminescence imaging.

In a third aspect, use of a compound of Formula I or Formula 2 in the calibration of distribution of a temperature-sensitive fluorescent compound when the temperature-sensitive fluorescent compound is used to measure the temperature distribution in a living cell is provided in the present invention,

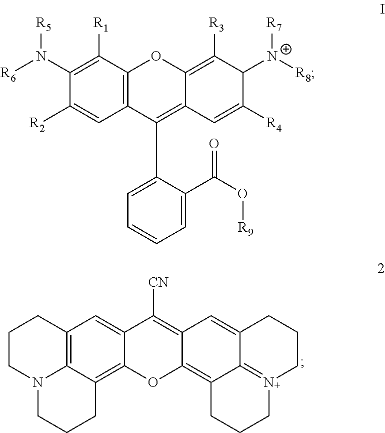

wherein,

R₉ is a hydrocarbyl of 1-22 carbon atoms, or an alkyl of 1 to 3 carbon atoms substituted with an ester group of 2-3 carbon atoms,

each of R₅, R₆, R₇, and R₈ is H, and

R₁, R₂, R₃, R₄ are H or a lower hydrocarbyl; or

R₉ is a hydrocarbyl of 1-22 carbon atoms, or an alkyl of 1 to 3 carbon atoms substituted with an ester group of 2-3 carbon atoms, and

R₅ and R₁, R₆ and R₂, R₇ and R₃, R₈ and R₄ are connected into a six-membered ring.

In a preferred embodiment, the hydrocarbyl can be an alkyl, alkenyl or alkynyl; preferably, a straight-chain or branched-chain or cyclic alkyl, e.g., methyl, ethyl, propyl, isopropyl, butyl, t-butyl, pentyl, cyclopentyl, cyclohexyl and the like; preferably, a straight-chain alkyl, such as methyl, ethyl, propyl, butyl, pentyl, and the like; more preferably, methyl or cetyl.

In a preferred embodiment, the alkyl group of 1 to 3 carbon atoms substituted by an ester group can be a methyl, ethyl or propyl, preferably methyl; and the ester group of 2-3 carbon atoms can be an ethyl ester group, propyl ester group.

In a preferred embodiment, the hydrocarbyl is a lower alkyl, alkenyl or alkynyl of 1-8 carbon atoms; preferably, an alkyl of 1-3 carbon atoms; more preferably, a methyl, ethyl or propyl.

In a specific embodiment, the compound is the following compound:

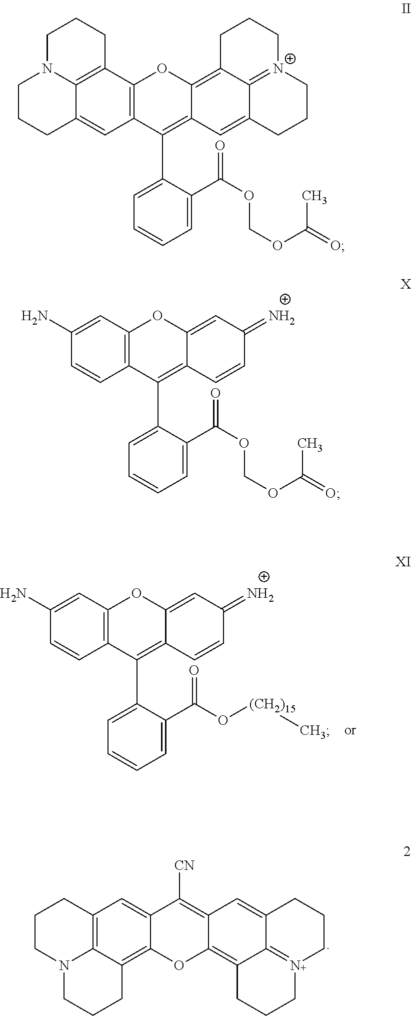

In a preferred embodiment, the use is to use the compound of Formula II or Formula X as a calibration material for the concentration distribution of the compound of Formula I when using the compound of Formula I to measure the temperature distribution of cytoplasm of a live cell.

In another preferred embodiment, the use is to use the compound of Formula XI as a calibration material for the concentration distribution of the compound of Formula I when using the compound of Formula I to measure the temperature distribution of cell membrane of a live cell.

In another preferred embodiment, the use is to use the compound of Formula 2 as a calibration material for the concentration distribution of the compound of Formula I when using the compound of Formula I to measure the temperature distribution of mitochondria of a live cell.

In another preferred embodiment, the use includes:

Calibration of distribution of the compound of Formula II: when the compound of Formula II is used to measure the temperature throughout the cytoplasm, Stokes luminescence of the compound itself is used for normalization;

Calibration of Rh101ME distribution: Anti-Stokes luminescence image produced by excited Rh101ME is normalized by using Stokes luminescence image of excited Rh800 (Formula 2);

Calibration of RhBAM distribution: Stokes luminescence image of excited RhBAM is normalized by using Stokes luminescence image of excited Rh110AM (Formula X);

Calibration of RhBME distribution: Stokes luminescence image of excited RhBME is normalized by using Stokes luminescence image of excited Rh800 (Formula 2);

Calibration of RhB-C16 distribution: Stokes luminescence image of excited RhB-C16 is normalized by using Stokes luminescence image of excited Rh110-C16 (Formula XI).

In a fourth aspect, the present invention provides a method for measuring temperature distribution in a living cell, said method including the following steps:

When anti-Stokes luminescence imaging of a temperature-sensitive fluorescent compound is used to measure the temperature,

(1) a compound of Formula I is used for staining living cells;

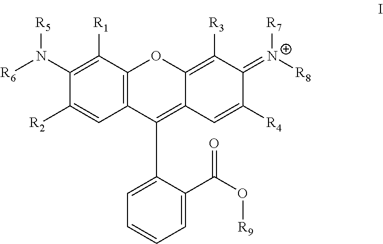

wherein,

R₉ is selected from the group consisting of a hydrocarbyl of 1-22 carbon atoms, an alkyl of 1 to 3 carbon atoms substituted with an ester group of 2-3 carbon atoms and an alkyl of 1 to 3 carbon atoms substituted with an aryl,

R₅, R₆, R₇, R₈ are independently selected from a hydrocarbyl, and

R₁, R₂, R₃, R₄ are H or a lower hydrocarbyl; or

R₉ is selected from the group consisting of a hydrocarbyl of 1-22 carbon atoms, an alkyl of 1 to 3 carbon atoms substituted with an ester group of 2-3 carbon atoms and an alkyl of 1 to 3 carbon atoms substituted with an aryl, and

R₅ and R₁, R₆ and R₂, R₇ and R₃, R₈ and R₄ are connected into a six-membered ring;

(2) the cells stained in step (1) are imaged under a fluorescence microscope;

(3) fluorescence image is calculated by using Equation (1):

Relative fluorescence intensity:

$$RI = A {e}^{- \frac{Δ E}{{k}_{B} T}} \qquad Equation ( 1 )$$

wherein k_{B} is Boltzmann's constant, T is the absolute temperature, ΔE is activation energy, A is fitting constants, and the relative fluorescence intensity is a ratio obtained from anti-Stokes luminescence of the compound of formula I normalized over Stokes luminescence of the compound itself,

standard curve of relative fluorescence intensity vs the temperature is pre-determined and Equation (1) is used for calculation, thereby obtaining the distribution image of temperature in living cells;

or

When Stokes luminescence or anti-Stokes luminescence imaging of a temperature-sensitive fluorescent compound is used to measure the temperature distribution within living cells,

(1) living cells are stained by a compound of formula I and a fluorescent compound used in calibration;

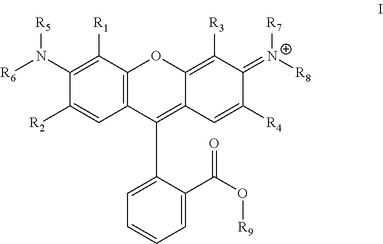

wherein,

R₉ is selected from the group consisting of a hydrocarbyl of 1-22 carbon atoms, an alkyl of 1 to 3 carbon atoms substituted with an ester group of 2-3 carbon atoms and an alkyl of 1 to 3 carbon atoms substituted with an aryl,

R₅, R₆, R₇, R₈ are independently selected from a hydrocarbyl, and

R₁, R₂, R₃, R₄ are H or a lower hydrocarbyl; or

R₉ is selected from the group consisting of a hydrocarbyl of 1-22 carbon atoms, an alkyl of 1 to 3 carbon atoms substituted with an ester group of 2-3 carbon atoms and an alkyl of 1 to 3 carbon atoms substituted with an aryl, and

R₅ and R₁, R₆ and R₂, R₇ and R₃, R₈ and R₄ are connected into a six-membered ring;

(2) the cells stained in step (1) are imaged under a fluorescence microscope;

(3) according to the linear relationship between the change in temperature and relative fluorescence intensity, a pre-determined standard curve is used for calculation to obtain a distribution image of temperature within living cells, wherein the relative fluorescence intensity refers to a ratio obtained from Stokes or anti-Stokes luminescence intensity of the temperature-sensitive fluorescent compound normalized by Stokes luminescence intensity of the fluorescent compound used in calibration.

In a preferred embodiment, R₅, R₆, R₇, R₈ are independently selected from an alkyl, alkenyl or alkynyl; in a further preferred embodiment, R₅, R₆, R₇, R₈ are independently selected from a lower alkyl; preferably, R₅, R₆, R₇, R₈ are independently selected from an alkyl of 1-8 carbon atoms; more preferably, R₅, R₆, R₇, R₈ are independently selected from from an alkyl of 1-3 carbon atoms; more preferably, R₅, R₆, R₇, R₈ are independently selected from methyl or ethyl; more preferably, all of R₅, R₆, R₇, R₈ are methyl or ethyl; and most preferably, all of R₅, R₆, R₇, R₈ are ethyl.

In a preferred embodiment, the lower hydrocarbyl is an alkyl, alkenyl or alkynyl of 1-8 carbon atoms; preferably, an alkyl of 1-3 carbon atoms; and more preferably, a methyl, ethyl or propyl.

In a preferred embodiment, a hydrocarbyl of 1-22 carbon atoms can be an alkyl, alkenyl or alkynyl of 1 to 22 carbon atoms; preferably, a straight or branched chain or cyclic alkyl of 1-22 carbon atoms, e.g., methyl, ethyl, propyl, isopropyl, butyl, t-butyl, pentyl, cyclopentyl, cyclohexyl and the like; preferably, a straight-chain alkyl, such as methyl, ethyl, propyl, butyl, pentyl, and the like; and more preferably, methyl or hexadecyl. The alkyl of 1-3 carbon atoms can be methyl, ethyl or propyl, preferably methyl; the ester group of 2-3 carbon atoms may be ethyl ester group, propyl ester group, preferably ethyl ester group; and the alkyl of 1-3 carbon atoms substituted by an aryl is methyl substituted by an aryl, ethyl substituted by an aryl or propyl substituted by an aryl; preferably, methyl substituted by an aryl; more preferably, methyl substituted by a substituent of formula IX

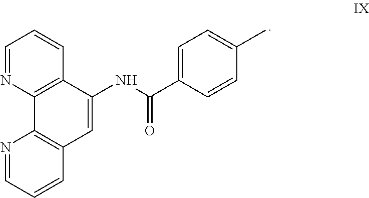 [SMILES: Cc1ccc(C(=O)Nc2cc3cccnc3c3ncccc23)cc1]

In another preferred embodiment, the calibration fluorescent compound is selected from a compound of Formula II, Formula X, Formula XI or Formula 2.

In a particular embodiment, the compound of formula I is a compound of the following formula:

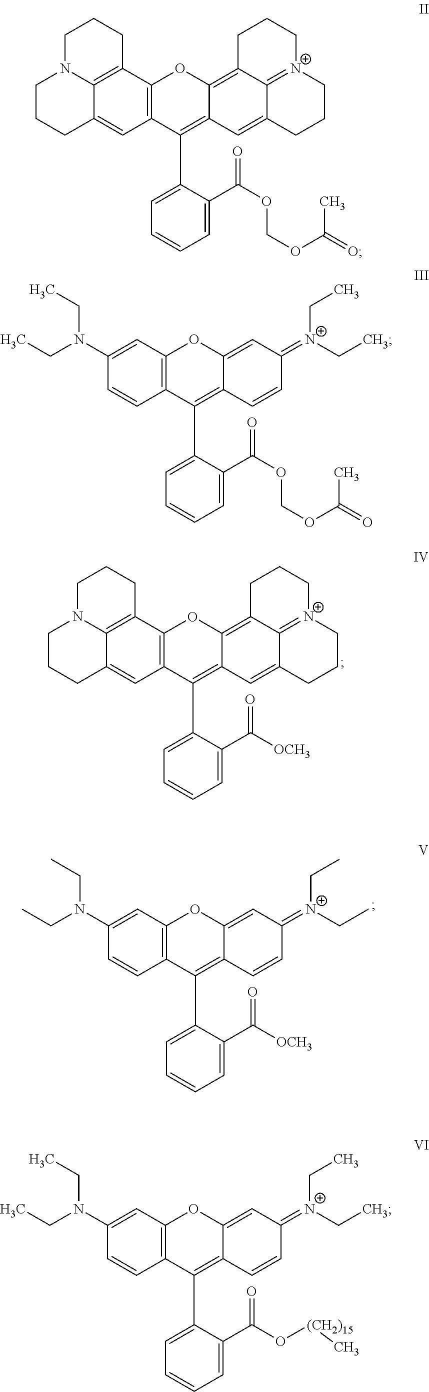

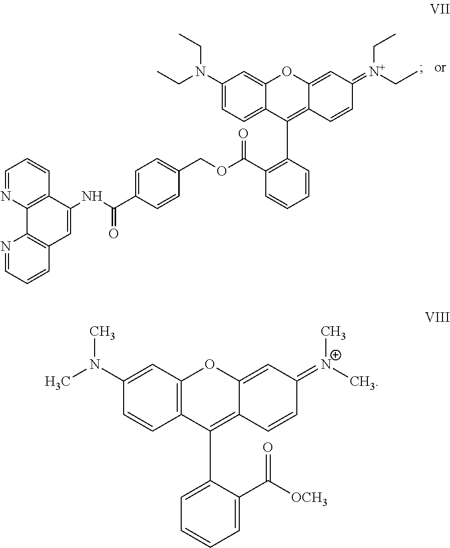

In a specific embodiment, the temperature distribution within a living cell is the temperature distribution in a subcellular structure; and preferably, the subcellular structure is cell membrane, cytoplasm, or mitochondria.

In another preferred embodiment, the method further includes inhibiting organic ion transporter by using an organic ion transporter inhibitor while measurement is performed.

In another preferred embodiment, the organic ion transporter inhibitor is probenecid, sulfinpyrazone or MK571.

In a fifth aspect, the present invention provides a calibration method for the distribution of a temperature-sensitive fluorescent compound when the temperature-sensitive fluorescent compound is used to measure the temperature distribution within a live cell, wherein another fluorescent compound without temperature-sensitive properties is used in the calibration of the distribution of the temperature-sensitive fluorescent compound, and said another fluorescent compound possesses the same intracellular concentration distribution as the temperature-sensitive fluorescent compound.

In a preferred embodiment, said another fluorescent compound without temperature-sensitive properties is covalently connected with said temperature-sensitive fluorescent compound. In another preferred embodiment, said another fluorescent compound without temperature-sensitive properties is covalently connected with said temperature-sensitive fluorescent compound through a hydrocarbon chain. In a more preferred embodiment, said another fluorescent compound without temperature-sensitive properties is covalently connected with said temperature-sensitive fluorescent compound through a hydrocarbon chain of 2-18 carbon atoms. In the most preferred embodiment, said another fluorescent compound without temperature-sensitive properties is covalently connected with said temperature-sensitive fluorescent compound through a hydrocarbon chain of 4-10 carbon atoms.

In a particular embodiment, the following compounds are used in the method for the calibration of distribution of the temperature-sensitive fluorescent compound:

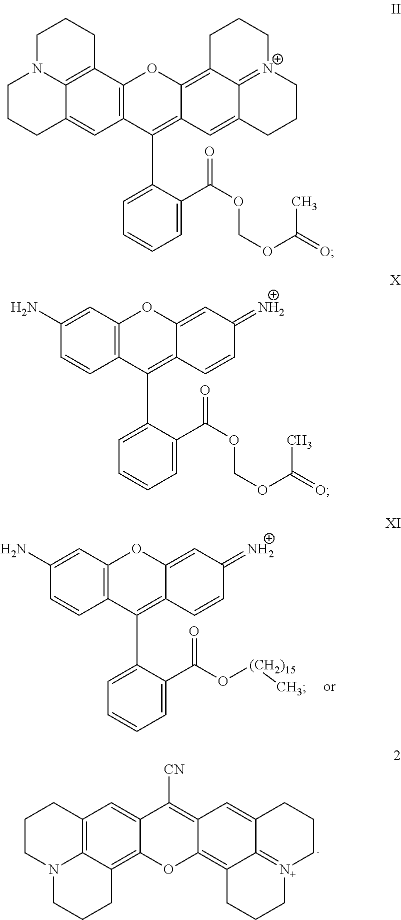

In a preferred embodiment, the calibration of distribution of temperature-sensitive fluorescent compounds includes:

the compound of Formula II or Formula X is used as a calibration material for the concentration distribution of the compound of Formula I when using the compound of Formula Ito measure the temperature distribution of cytoplasm of a live cell;

the compound of Formula XI is used as a calibration material for the concentration distribution of the compound of Formula I when using the compound of Formula Ito measure the temperature distribution of cell membrane of a live cell;

the compound of Formula 2 is used as a calibration material for the concentration distribution of the compound of Formula I when using the compound of Formula I to measure the temperature distribution of mitochondria of a live cell.

In another preferred embodiment, the calibration of distribution of temperature-sensitive fluorescent compounds includes:

Calibration of distribution of the compound of Formula II: when the compound of Formula II is used to measure the temperature throughout the cytoplasm, Stokes luminescence of the compound itself is used for normalization;

Calibration of Rh101ME distribution: Anti-Stokes luminescence image produced by excited Rh101ME is normalized by using Stokes luminescence image of excited Rh800 (Formula 2);

Calibration of RhBAM distribution: Stokes luminescence image of excited RhBAM is normalized by using Stokes luminescence image of excited Rh110AM (Formula X);

Calibration of RhBME distribution: Stokes luminescence image of excited RhBME is normalized by using Stokes luminescence image of excited Rh800 (Formula 2);

Calibration of RhB-C16 distribution: Stokes luminescence image of excited RhB-C16 is normalized by using Stokes luminescence image of excited Rh110-C16 (Formula XI).

In a sixth aspect, the present invention provides a kit for measuring the temperature distribution within a living cell, the kit comprising:

(1) a compound of formula I:

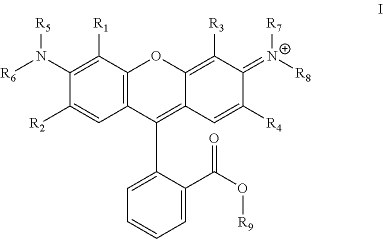

wherein,

R₉ is selected from the group consisting of a hydrocarbyl of 1-22 carbon atoms, an alkyl of 1 to 3 carbon atoms substituted with an ester group of 2-3 carbon atoms and an alkyl of 1 to 3 carbon atoms substituted with an aryl,

R₅, R₆, R₇, R₈ are independently selected from a hydrocarbyl, and

R₁, R₂, R₃, R₄ are H or a lower hydrocarbyl; or

R₉ is selected from the group consisting of a hydrocarbyl of 1-22 carbon atoms, an alkyl of 1 to 3 carbon atoms substituted with an ester group of 2-3 carbon atoms and an alkyl of 1 to 3 carbon atoms substituted with an aryl, and

R₅ and R₁, R₆ and R₂, R₇ and R₃, R₈ and R₄ are connected into a six-membered ring;

(2) auxiliary reagents used in cell staining;

(3) containers for accomodating the above compounds and auxiliary reagents; and

(4) instruction manual for using said compound to measure temperature distribution inside a living cell.

In a specific embodiment, the compound is the following compound:

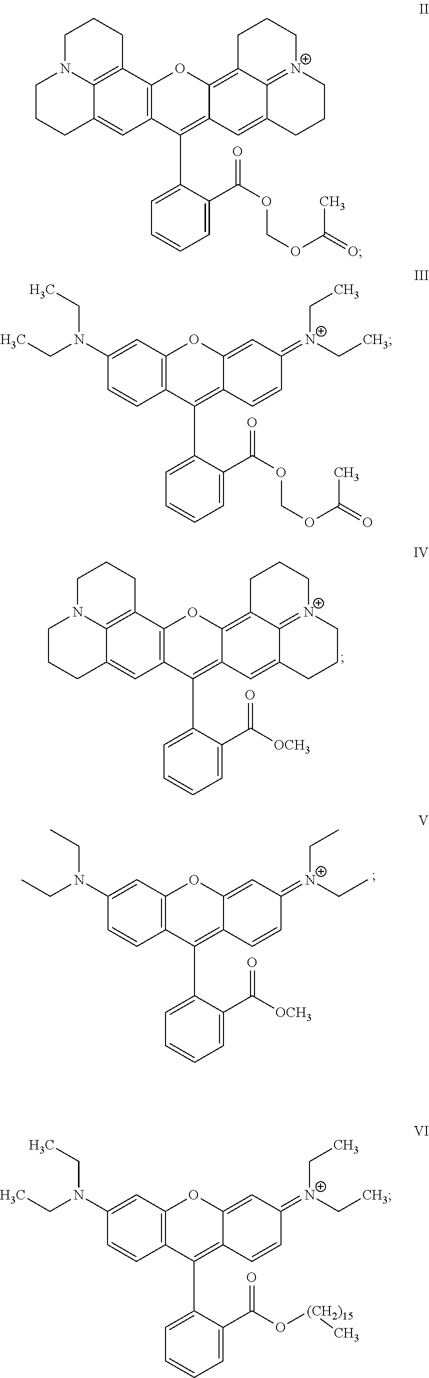

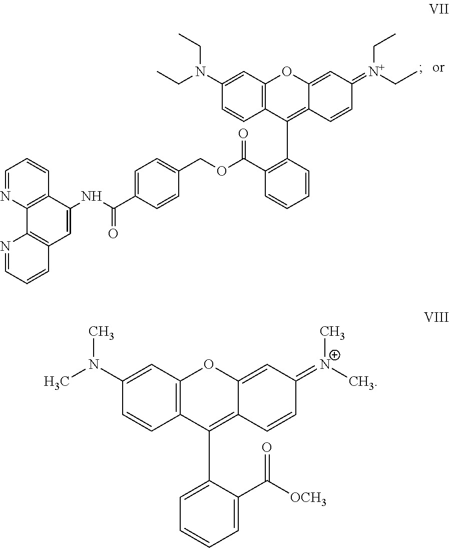

In another specific embodiment, the detection kit also comprises the following compounds:

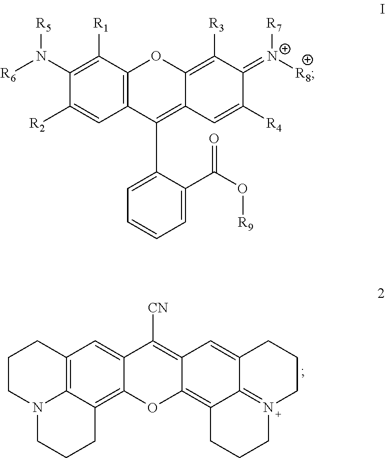

wherein,

R₉ is a hydrocarbyl of 1-22 carbon atoms, or an alkyl of 1 to 3 carbon atoms substituted with an ester group of 2-3 carbon atoms,

all of R₅, R₆, R₇, R₈ are H, and

all of R₁, R₂, R₃, R₄ are H or a lower hydrocarbyl; or

R₉ is a hydrocarbyl of 1-22 carbon atoms, or an alkyl of 1 to 3 carbon atoms substituted with an ester group of 2-3 carbon atoms, and

R₅ and R₁, R₆ and R₂, R₇ and R₃, R₈ and R₄ are connected into a six-membered ring.

In another specific embodiment, the compound is the following compound:

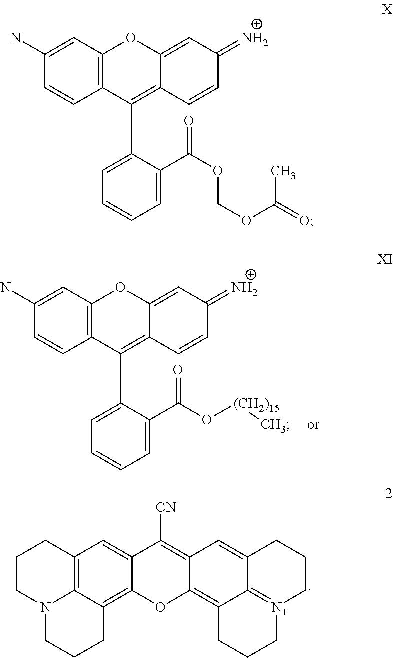

In a preferred embodiment, the temperature distribution within a living cell is the temperature distribution in a subcellular structure; and preferably, the subcellular structure is cell membrane, cytoplasm, or mitochondria.

In another preferred embodiment, the measurement of temperature distribution within a living cell is the measurement of temperature distribution of cytoplasm within a living cell by using the compound of formula II or formula III for measuring.

In another preferred embodiment, the measurement of temperature distribution within a living cell is the measurement of temperature distribution of mitochondria within a living cell by using the compound of formula IV or formula V.

In another preferred embodiment, the measurement of temperature distribution within a living cell is the measurement of temperature distribution of cell membrane of a living cell by using the compound of formula VI.

In another preferred embodiment, the measurement of temperature distribution within a living cell is the measurement of temperature of mitochondria within a living cell by using the compound of formula VII, VIII.

It should be understood that in the present invention, the technical features specifically mentioned above and below (such as in the Examples) can be combined with each other, thereby constituting a new or preferred technical solution which needs not be individually described.

### Terms Used in the Present Invention

The present invention relates to two types of fluorescence, ie., Stokes and anti-Stokes luminescence.

Stokes luminescence is commonly known as fluorescence, which is characterised as a shift of fluorescence spectrum toward long wavelength direction (redshift) compared with the corresponding absorption spectra.

Anti-Stokes luminescence refers to a shift of fluorescence spectrum toward short wavelength direction (blue shift) compared with the corresponding absorption spectra.

When light hits a molecule and interacts with electron cloud and molecule bonding, the molecule can be excited from ground state to a virtual energy state (excited state), thereby producing Stokes and anti-Stokes luminescence. When the molecule in excited state emits a photon and returns to a rotating or vibrating state which is different from the ground state, the frequency of the emitted photon will be different from the wavelength of the excitation light due to the energy difference between the ground state and the new state. If the energy of the molecule in the final vibrational state is higher than that in initial state, the frequency of the emitted photon is lower (i.e., longer wavelength) to ensure the conservation of total energy of the system. Such a change in frequency is named as Stokes shift, and fluorescence generated during this process is Stokes luminescence. If the energy of the molecule in the final vibrational state is lower than that in initial state, the frequency of the emitted photon is higher (i.e., shorter wavelength). Such a change in frequency is named as anti-Stokes shift, and fluorescence generated during this process is anti-Stokes luminescence.

Relative Fluorescence Intensity refers to a ratio obtained from normalization process of light intensity of a temperature-sensitive fluorescent compound against a non-temperature-sensitive fluorescent compound when the temperature-sensitive fluorescent compound is used to measure intracellular temperature, wherein the concentration distribution of the non-temperature-sensitive fluorescent compound is consistent with that of the temperature-sensitive fluorescent compound; and also refers to a ratio obtained from normalization process of anti-Stokes luminescent without temperature-sensitive property of fluorescent compound against Stokes luminescent with temperature-sensitive property of the fluorescent compound.

In the present invention, “Rh” is an abbreviation of “Rhodamine”.

### BRIEF DESCRIPTION OF DRAWINGS

FIG. 1 shows a spectrum of Rh101 and derivatives thereof. Wherein la shows excitation spectrum (dotted line, light emission collected at 640 nm) and emission spectrum (solid line, excited at 530 nm) of Rh101 (blackcurve), Rh101AM (green curves), Rh101ME (red curve). Concentration of dye is 10 μM, and solvent is 150 mM KCl solution, pH 7.5; 1b shows anti-Stokes emission spectra (excited at 633 nm) of 10 μM Rh101 (dissolved in 150 mM KCl solution at pH 7.5) at different temperatures (from top to bottom, 45, 35, 25, 15, 5□ respectively), and Stokes luminescence intensity is normalized against peak at 25□; (c) excitation spectrum (dotted line, light emission collected at 640 nm) and Stokes emission spectrum (solid line, excited at 530 nm) of Rh101ME. Concentration of dye is 10 μM, and solvent is 150 mM KCl solution, pH 7.5; (d) anti-Stokes emission spectra (excited at 633 nm) of 10 μM Rh101ME (dissolved in 150 mM KCl solution at pH 7.5) at different temperatures (from top to bottom, 45, 35, 25, 15, 5□ respectively), and anti-Stokes luminescence intensity is normalized against peak at 25□.

FIG. 2 shows spectroscopic properties of RhB and derivatives thereof, and the emission intensities of Stokes luminescent of them are in negative linear correlation with temperature. Wherein, 2a shows excitation spectrum (dotted line, light emission collected at 640 nm) and emission spectrum (solid line, excited at 530 nm) of RhB (black curve), RhBAM (green curve), RhBME (red curve). Concentration of dye is 10 μM, and solvent is 150 mM KCl solution, pH 7.5; 2b shows emission spectra (excited at 530 nm) of 10 μM RhB (dissolved in 150 mM KCl solution at pH 7.5) at different temperatures (from top to bottom, 45, 35, 25, 15, 5□ respectively), and Stokes luminescence intensity is normalized against peak at 25□; (c) excitation spectrum (dotted line, light emission collected at 640 nm) and emission spectrum (solid line, excited at 530 nm) of RhBME. Concentration of dye is 10 μM, and solvent is 150 mM KCl solution, pH 7.5; (d) Stokes emission spectra (excited at 530 nm) of 10 μM RhBME (dissolved in 150 mM KCl solution at pH 7.5) at different temperatures (from bottom to top, 45, 35, 25, 15, 5□ respectively), and Stokes luminescence intensity is normalized against peak at 25□.

FIG. 3 shows Stokes luminescence image of HepG2 cells captured by EMCCD (Evolve 512, Photometrice Ltd.) under fluorescence microscope (BX61WI, Olympus Ltd., 40×lens, numerical aperture NA 0.8, the temperature of culture solution is 27.9□ at imaging), after the cells were stained by using 200 nM Rh101AM for 60 mins in an incubator at 37□. Wherein, 3a shows Stokes luminescence image from a monochromator (Optoscan monochromator, Cairn Research Ltd.), excited at a wavelength of 555 nm (bandwidth of 3 nm) and collected at 573˜613 nm; 3b shows anti-Stokes luminescence image from a monochromator, excited at a wavelength of 635 nm (bandwidth of 15 nm) and collected at 573˜613 nm; 3c shows a ratio image obtained from normalizing FIG. 3b over FIGS. 3a; and 3d shows the temperature distribution of cells calculated according to equation (1);

FIG. 4 shows Stokes luminescence image of HepG2 cells captured by EMCCD (Evolve 512, Photometrice Ltd.) under fluorescence microscope (BX61WI, Olympus Ltd., 40×lens, numerical aperture 0.8, the temperature of culture solution is 27.9□ at imaging), after the cells were stained by using 200 nM Rh101 for 60 mins in an incubator at 37□. Wherein, 4a shows Stokes luminescence image from a monochromator (Optoscan monochromator, Cairn Research Ltd.), excited at a wavelength of 555 nm (bandwidth of 3 nm) and collected at 573˜613 nm; and 4b shows anti-Stokes luminescence image from a monochromator, excited at a wavelength of 635 nm (bandwidth of 15 nm) and collected at 573˜613 nm.

FIG. 5 shows Stokes luminescence image of HepG2 cells captured by EMCCD (Evolve 512, Photometrice Ltd.) under fluorescence microscope (BX61WI, Olympus Ltd., 40×lens, numerical aperture 0.8, the temperature of culture solution is 27.9° C. at imaging) at different perfusion time points, after the cells were stained by using 200 nM Rh101AM or Rh101 for 60 mins in an incubator at 37° C. Wherein, 5a-c show Stokes luminescence image from a monochromator, excited at a wavelength of 555 nm (bandwidth of 3 nm) and collected at 573˜613 nm, respectively, after cells were stained with Rh101AM and lavaged with 2.5 mM probenecid perfusion solution (Tyrode solution) for 0 min, 10 min, 20 min; 5d-f show Stokes luminescence image from a monochromator, excited at a wavelength of 555 nm (bandwidth of 3 nm) and collected at 573˜613 nm, respectively, after cells were stained with Rh101AM and lavaged with perfusion solution without probenecid (Tyrode solution) for 0 min, 10 min, 20 min; 5g-i show Stokes luminescence image from a monochromator, excited at a wavelength of 555 nm (bandwidth of 3 nm), respectively, after cells were stained with Rh101 and lavaged with 2.5 mM probenecid perfusion solution (Tyrode solution) for 0 min, 10 min, 20 min; and 5j shows curves of Stokes luminescence intensity vs perfusion time under the above 3 conditions, wherein in each condition normalization was performed against result of perfusion at 0 min.

FIG. 6 shows image of COS7 cells under laser confocal fluorescence microscope (FV1000, Olympus, 60×water immersed lens, numerical aperture 1.2, the temperature of culture solution is 30° C. at imaging), after the cells were stained by using 100 nM Rh101ME and 100 nM Rh800 for 30 mins in an incubator at 37□. Wherein 6a shows Stokes luminescence image produced by Rh800 excited by a laser at 635 nm and collected at 655˜755 nm; 6b shows anti-Stokes luminescence image produced by Rh101ME excited by a laser at 635 nm and collected at 575˜620 nm; and 6c shows temperature distribution image of mitochondria obtained through calculation.

FIG. 7 shows image of COS7 cells under laser confocal fluorescence microscope (FV1000, Olympus, 60×water immersed lens, numerical aperture 1.2, the temperature of culture solution is 30° C. at imaging), after the cells were stained by using 50 nM RhBME and 50 nM Rh800 for 30 mins in an incubator at 37□. Wherein 7a shows Stokes luminescence image produced by RhBME excited by a laser at 559 nm and collected at 575˜620 nm; and 7b shows Stokes luminescence image produced by Rh800 excited by a laser at 635 nm and collected at 655˜755 nm; and 7c shows temperature distribution image of mitochondria obtained through calculation.

FIG. 8 shows temperature sensitive properties and cell-membrane localization properties of RhB-C16 compound (a compound of formula VI). Wherein 8a shows excitation spectrum (dotted line, light emission collected at 640 nm) and emission spectrum (solid line, excited at 530 nm) of RhB-C16. Concentration of dye is 10 μM, and solvent is DMSO; 8b shows emission spectra (excited at 530 nm) of 10 μM RhB-C16 (dissolved in DMSO) at different temperatures (from top to bottom, 25, 35, 45, 55□ respectively), and Stokes luminescence intensity is normalized against peak at 25□; and 8c shows that RhB-C16 (compound of Formula VI) is located on the cell membrane, and 8c also shows image of HepG2 cells under fluorescence microscope (FV1000, Olympus, 20×lens, numerical aperture 0.8, the temperature of culture solution is 20° C. at imaging), after the cells were stained by using 1 μM RhB-C16 for 5 mins in an incubator at 37° C., which is Stokes luminescence image excited by laser at 559 nm and collected at 575˜620 nm.

FIG. 9 shows that RPA compound (a compound of formula VII) possesses temperature sensitive properties. Wherein, 9a shows the excitation spectrum (dotted line, light emission collected at 640 nm) and emission spectrum (solid line, excited at 530 nm) of RAP. Concentration of dye is 10 μM, and solvent is DMSO; and 9b shows emission spectra (excited at 530 nm) of 10 μM RPA (dissolved in DMSO) at different temperatures (from top to bottom, 25, 35, 45, 55° C. respectively), and Stokes luminescence intensity is normalized against peak at 25° C.

FIG. 10 shows that TMRM compound (a compound of formula VIII) possesses temperature sensitive properties. Wherein, 10a shows excitation spectrum (dotted line, light emission collected at 640 nm) and emission spectrum (solid line, excited at 530 nm) of TMRM. Concentration of dye is 10 μM, and solvent is 150 mM KCl solution, pH 7.5; and 10b shows emission spectra (excited at 530 nm) of 10 μM RhB (dissolved in 150 mM KCl solution, pH 7.5) at different temperatures (from bottom to top, 45, 35, 25, 15, 5° C. respectively), and Stokes luminescence intensity is normalized against peak at 25° C.

FIG. 11 shows that Stokes luminescence of Rh110 compound does not possess temperature sensitive properties. Wherein 11(a) shows excitation spectrum (dotted line, light emission collected at 555 nm) and emission spectrum (solid line, excited at 470 nm) of Rh110. Concentration of dye is 10 μM, and solvent is 150 mM KCl solution, pH 7.5; and (b) shows emission spectra (excited at 470 nm) of 10 μM Rh110 (dissolved in 150 mM KCl solution, pH 7.5) at different temperatures (45, 35, 25, 15, 5° C. respectively), and Stokes luminescence intensity is normalized against peak at 25° C.

FIG. 12 shows that Stokes luminescence of Rh110 compound does not possess temperature sensitive properties. Wherein 12(a) shows excitation spectrum (dotted line, light emission collected at 640 nm) and emission spectrum (solid line, excited at 530 nm) of Rh101. Concentration of dye is 10 μM, and solvent is 150 mM KCl solution, pH 7.5; and (b) shows Stokes emission spectra (excited at 530 nm) of 10 μM Rh101 (dissolved in 150 mM KCl solution, pH 7.5) at different temperatures (45, 35, 25, 15, 5° C. respectively), and Stokes luminescence intensity is normalized against peak at 25° C.

FIG. 13 shows that Stokes luminescence of Rh800 compound does not possess temperature sensitive properties. Wherein 12(a) shows excitation spectrum (dotted line, light emission collected at 750 nm) and emission spectrum (solid line, excited at 635 nm) of Rh800. Concentration of dye is 10 μM, and solvent is 150 mM KCl solution, pH 7.5; and (b) shows Stokes emission spectra (excited at 635 nm) of 10 μM Rh800 (dissolved in 150 mM KCl solution, pH 7.5) at different temperatures (45, 35, 25, 15, 5° C. respectively), and Stokes luminescence intensity is normalized against peak at 25° C.

FIG. 14 (a)-(e) show the HNMR spectrum of compounds of Formula II, III, IV, V, VI, and (f) shows partially enlarged view of HNMR spectrum of the compound of Formula VI.

### EMBODIMENTS FOR CARRYING OUT THE INVENTION

A cell is in the size of μm, and the changes in temperature in a single cell will rapidly affected by the ambient solution, therefore, it is generally difficult for a conventional method for measuring temperature to detect temperature distribution within a living cell. Anti-Stokes emission intensity of fluorescent dye rhodamine 101 (Rh101) is enhanced as the temperature increases, while Stokes emission intensity of Rhodamine B (RhB) is decreased as the temperature increases [2, 3]. In the prior art, both of the dyes are used to measure the temperature inside a cell or tissue samples based on their temperature sensitive properties [4, 5]. However, during the study, the present inventors have unexpectedly discovered that neither of the compounds can effectively permeate through a cell membrane and enter into the cell, therefore, the measured temperature is not the temperature within the cell. However, the literatures mentioned above do not notice or suggest the presence of the problem or defect. To accurately and rapidly measure intracellular temperature, a temperature-sensitive fluorescent dye which can penetrate cell membrane and enter into a cell is necessary.

Through extensive and in-depth study, the inventors have unexpectedly found that derivatives obtained from rhodamine 101 (Rh101) and rhodamine B (RhB) through structural modification can penetrate cell membrane, and even can be enriched in mitochondria. Therefore it is convenient to stain cells, and in turn observe distribution and variation of temperature in a cell and mitochondria. Based on the above findings, the present invention is completed.

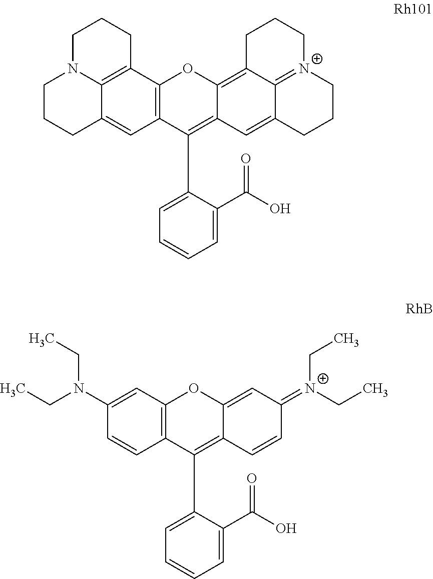 [SMILES: O=C(O)c1ccccc1C1=c2cc3c4c(c2Oc2c1cc1c5c2CCCN5CCC1)CCC[N+]=4CCC3 ; CCN(CC)c1ccc2c(-c3ccccc3C(=O)O)c3ccc(=[N+](CC)CC)cc-3oc2c1]

### COMPOUNDS OF THE PRESENT INVENTION

To solve the above problems present in the prior art, a compound of formula I is provided in the present invention:

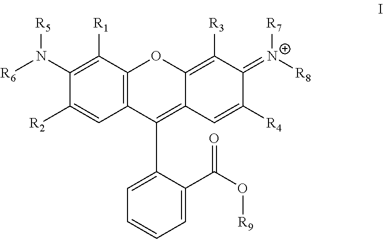

Wherein,

R₉ is selected from the group consisting of a hydrocarbyl of 1-22 carbon atoms, an alkyl of 1 to 3 carbon atoms substituted with an ester group of 2-3 carbon atoms and an alkyl of 1 to 3 carbon atoms substituted with an aryl,

R₅, R₆, R₇, R₈ are independently selected from a hydrocarbyl or H, and

R₁, R₂, R₃, R₄ are H or a lower hydrocarbyl; or

R₉ is selected from the group consisting of a hydrocarbyl of 1-22 carbon atoms, an alkyl of 1 to 3 carbon atoms substituted with an ester group of 2-3 carbon atoms and an alkyl of 1 to 3 carbon atoms substituted with an aryl, and

R₅ and R₁, R₆ and R₂, R₇ and R₃, R₈ and R₄ are connected into a six-membered ring.

A person skilled in the art will know that, as used herein, the term “hydrocarbyl” means a straight-chain or branched-chain, saturated or unsaturated group consisting of C and H; in particular, an alkyl, alkenyl or alkynyl. In a preferred embodiment, the lower hydrocarbyl is an alkyl alkenyl or alkynyl of 1-8 carbon atoms; preferably, an alkyl of 1-3 carbon atoms; more preferably, a methyl, ethyl or propyl.

In a preferred embodiment, each of R₅, R₆, R₇, and R₈ is independently selected from an alkyl, alkenyl, alkynyl or H; in a further preferred embodiment, each of R₅, R₆, R₇, and R₈ is independently selected from a lower alkoxy or H; preferably, each of R₅, R₆, R₇, and R₈ is independently selected from an alkyl of 1 to 8 carbon atoms or H; more preferably, each of R₅, R₆, R₇, and R₈ is independently selected from an alkyl of 1 to 3 carbon atoms or H; more preferably, each of R₅, R₆, R₇, and R₈ is independently selected from methyl or ethyl or H; more preferably, all of R₅, R₆, R₇, and R₈ are methyl or ethyl or H; and most preferably, all of R₅, R₆, R₇, R₈ are ethyl or H.

In a preferred embodiment, a hydrocarbyl of 1-22 carbon atoms can be an alkyl, alkenyl or alkynyl of 1 to 22 carbon atoms; preferably, a straight or branched chain or cyclic alkyl of 1-22 carbon atoms, e.g., methyl, ethyl, propyl, isopropyl, butyl, t-butyl, pentyl, cyclopentyl, cyclohexyl and the like; preferably, a straight-chain alkyl, such as methyl, ethyl, propyl, butyl, pentyl, and the like; and more preferably, methyl or hexadecyl; the alkyl of 1-3 carbon atoms can be methyl, ethyl or propyl, preferably methyl; the ester group of 2-3 carbon atoms can be an ethyl ester group, propyl ester group, preferably ethyl ester group; the alkyl of 1-3 carbon atoms substituted by an aryl is a methyl substituted by an aryl, ethyl substituted by an aryl or propyl substituted by an aryl; preferably a methyl substituted by an aryl; and more preferably an methyl substituted by a substituent of formula IX

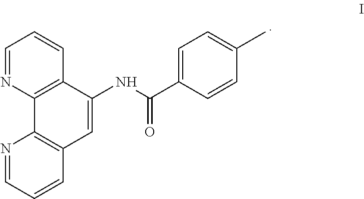 [SMILES: Cc1ccc(C(=O)Nc2cc3cccnc3c3ncccc23)cc1]

Accordingly, the present invention provides the following specific compounds:

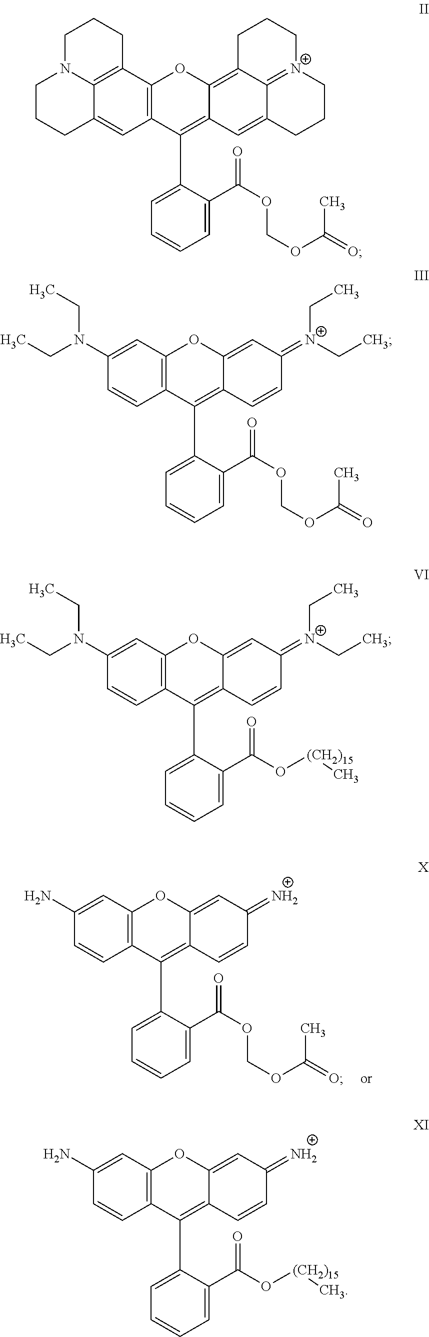

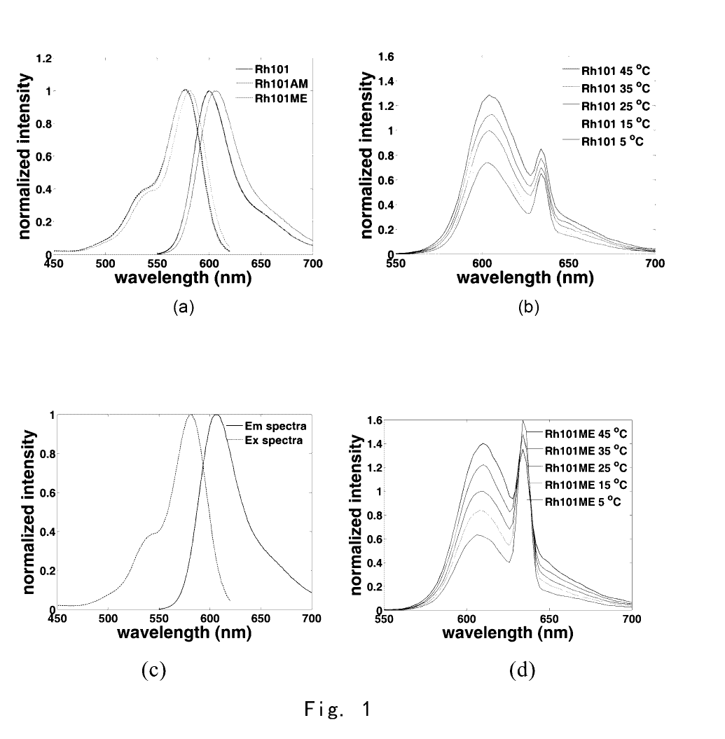

From the spectrogram of Rh101 and derivatives thereof (FIG. 1a), it can be seen that there is no significant difference in spectroscopic characteristics between the compound of formula II (Rh101AM) or the compound of formula IV (Rh101ME) and Rh101. The anti-Stokes luminescence intensity of Rh101 increases as the temperature increases (FIG. 1B).

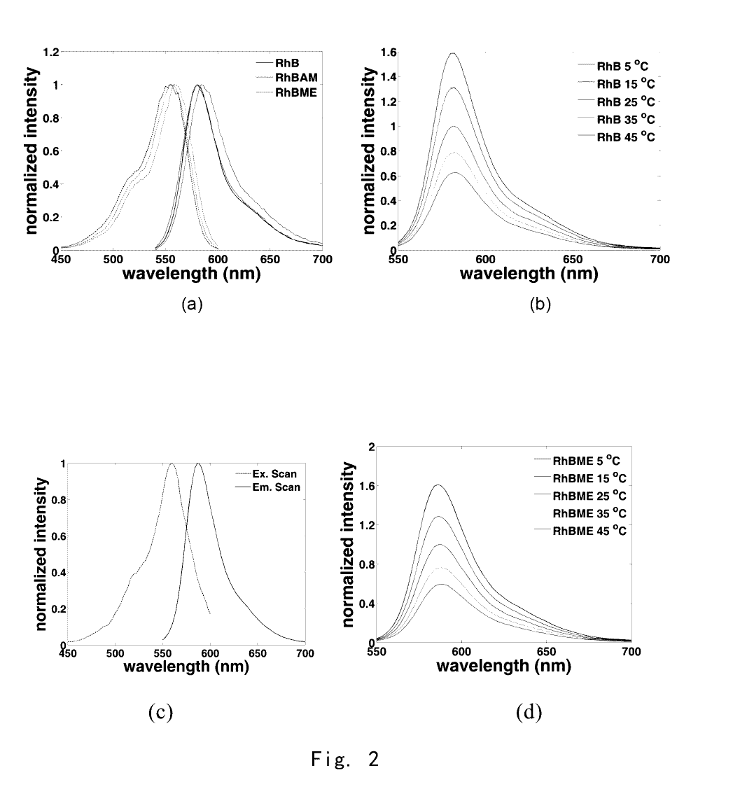

And spectroscopic characteristics of RhB derivatives (the compound of formula III and V) are consistent with those of RhB (FIG. 2), and the Stokes luminescence intensity of them decreases as the temperature increases.

### Use of the Compounds According to the Present Invention in the Measurement of Temperature Distribution Inside a Living Cell

Since Stokes luminescence intensity or anti-Stokes luminescence intensity of the compound of formula I provided in the present invention is relevant to the temperature, and the compound can penetrate cell membrane, and even can be enriched in subcellular structure, such as cytoplasm, cell membrane and mitochondria, it is more convenient to stain cells. Therefore, the compound of formula I according to the present invention can be used to measure temperature distribution in a living cell.

As described herein, temperature distribution in a living cell refers to the temperature distribution of subcellular structures; and subcellular structure refers to part of the cell structure, generally smaller than a cell, including but not limited to cell membrane, mitochondria, centrosome, Golgi, cytoplasm, and the like. In a preferred embodiment, the subcellular structure is cell membrane, cytoplasm, or mitochondria. Subcellular localization as described herein refers to distribution of a fluorescent compound on said subcellular structures.

In a particular embodiment, the compound of Formula II or Formula III according to the present invention can be used to measure temperature distribution of cytoplasm within a living cell. In another specific embodiment, the compound of Formula IV or Formula V according to the present invention can be used to measure temperature distribution of mitochondria within a living cell. In another specific embodiment, the compound of Formula VI according to the present invention can be used to measure temperature distribution of cell membrane within a living cell. In another specific embodiment, the compound of Formula VII, Formula VIII according to the present invention can be used to measure temperature distribution of mitochondria within a living cell.

### Distribution Calibration of Temperature-Sensitive Fluorescent Compound (Normalization)

Fluorescence intensity of a temperature-sensitive fluorescent compound is not only relevant to temperature, but also to local concentration of the compound. After entering cells and mitochondria, distribution of the fluorescent compound may be inhomogeneous, therefore, fluorescence emitted by fluorescent compounds, accumulation of which in a cell is different, can not be compared with each other.

When anti-Stokes luminescence of a temperature-sensitive fluorescent compound is used to detect temperature distribution within a living cell, if Stokes luminescence of the compound does not change with the temperature, it can be used to represent concentration distribution of the compound. Influence of concentration on fluorescence intensity can be eliminated by normalizing anti-Stokes luminescence intensity against Stokes luminescence intensity, and the ratio thus obtained is named as relative fluorescence intensity, the variation of which is in line with Maxwell-Boltzmann statistics and can be fitted by using equation (1) [3]:

relative fluorescence intensity:

$$RI = A {e}^{- \frac{Δ E}{{k}_{B} T}} , \qquad equation ( 1 )$$

wherein k_{B} is Boltzmann constant, T is absolute temperature, ΔE is activation energy, and A is fitting constant.

Ratio image (i.e., image of relative fluorescence intensity) can be obtained by normalizing anti-Stokes luminescence image of a fluorescent compound using its Stokes luminescence image. And the image of temperature distribution can be obtained through calculation using pre-determined standard curve [4]. However, when using such method in calibration, there is time difference between collected Stokes luminescence and anti-Stokes luminescence signal, resulting in inaccurate calculated temperature, since one compound is excited by two different exciting lights and it is impossible to excite the compound at the same time. The error due to such time difference is tolerable for determining temperature distribution of a large-scale range, such as cytoplasm. However, measurement of temperature for a fine structure, such as an organelle (such as mitochondria), will be greatly influenced.

To eliminate the error due to time difference in the above calibration method, upon intensive study, the present inventors found that another fluorescent compound can be used in distribution calibration of a temperature-sensitive fluorescent compound used to measure the temperature distribution within a living cell, wherein said another fluorescent compound possesses the same intracellular concentration distribution as the temperature-sensitive fluorescent compound and does not possess temperature-sensitive properties. By carefully selecting the wavelength of exciting light and the wavelength of the collected fluorescence signal, the temperature-sensitive fluorescent compound and calibration fluorescent compound can be simultaneously excited, fluorescence signals can be simultaneously collected, and there won't be time difference between two fluorescent signals, therefore, intracellular temperature can be more accurately measured.

Accordingly, a method for the calibration of the distribution of a temperature-sensitive fluorescent compound is provided in the present invention, wherein another fluorescent compound (calibration compound) is used in distribution calibration of a temperature-sensitive fluorescent compound, wherein said another fluorescent compound possesses the same intracellular concentration distribution as the temperature-sensitive fluorescent compound and does not possess temperature-sensitive properties.

The present inventors have further found that a temperature-sensitive fluorescent compound can be covalently attached to another fluorescent compound without temperature-sensitive properties, so that the concentration distribution and kinetic properties of two fluorescent compounds are completely identical, thereby further eliminating the error due to differences in concentration or differences in dynamics properties. In a preferred embodiment, a temperature-sensitive fluorescent compound is covalently attached to another fluorescent compound without temperature sensitive properties as said above via a hydrocarbon chain; more preferably, covalently attached through a hydrocarbon chain of 2-18 carbon atoms; most preferably, covalently attached through a hydrocarbon chain of 4-10 carbon atoms. Based on routine technical means in the field of chemical synthesis, such covalent attachment can be achieved by a skilled person in the art according to the specific structure of a compound and employing a variety of suitable methods and linker. For example, diol of 2-18 carbon atoms can be used to covalently link a temperature-sensitive fluorescent compound with an ester bond or a carboxyl to a calibration compound through transesterification reaction or esterification reaction:

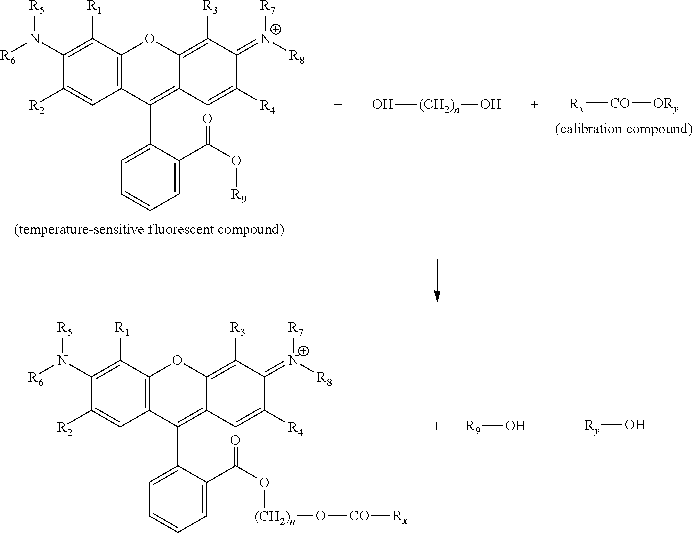

(n is a natural number from 2-18)

In a specific embodiment, the following compounds are used in the distribution calibration for a temperature-sensitive fluorescent compound:

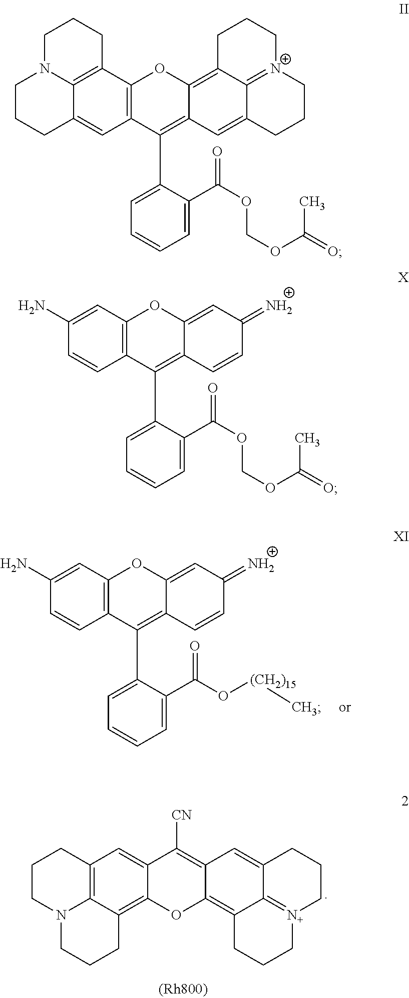

In a specific embodiment, the distribution calibration program is:

Distribution calibration of Rh101AM (compound of Formula II): Stokes luminescence intensity of Rh101AM does not substantially change as the temperature changes, thus can be used to represent the concentration distribution of the dye, and can be used to normalize anti-Stokes luminescence image of Rh101AM, thereby obtaining the distribution image of temperature.

Distribution calibration of Rh101ME (compound of Formula IV): upon careful selection and numerous experiments, the inventors discovered that Rh800 (the compound of Formula 2) distributes on mitochondria as Rh101ME does, and Stokes luminescence intensity of Rh800 does not substantially change as the temperature changes at a wavelength range of less than 700 nm, therefore, the concentration of Rh800 can be used to indirectly reflect the concentration distribution of Rh101ME. Stokes luminescence image obtained by simultaneously using Rh101ME and Rh800 to stain cells, exciting Rh800 with a laser at wavelength of 635 nm and collecting luminescence at 655˜755 nm can reflect the concentration distribution of two dyes. The image can be used to normalize anti-Stokes luminescence image of Rh101ME obtained by exciting with a laser at wavelength of 635 nm and collecting luminescence at 575˜620 nm, thereby obtaining a ratio image reflecting the temperature distribution of a sample. Since in this program, the same exciting light is used and luminescence is collected at different ranges of emitted light, there is not time difference between the two collected fluorescent signals. Chronologically, Stokes luminescence image of Rh800 and anti-Stokes luminescence image of Rh101ME can exactly match.

Distribution calibration of RhBAM (compound of Formula III): a ratio image of RhBAM can also be obtained according to the same program as that for Rh101ME to measure the temperature of cytoplasm or mitochondria. Upon several experiments, the inventors discovered that Rh110 AM (the compound of formula X) is suitable for calibration of intracelluar distribution of RhBAM. Rh110 is a green fluorescent dye, Stokes luminescence intensity of which is not sensitive to changes in temperature. Intracellular distribution of synthesized Rh110AM is identical with that of RhBAM, both of which are in cytoplasm, and the range of emitted light and exciting light between them is different. Stokes luminescence image obtained by exciting Rh110AM using a laser with a wavelength of 488 nm, and collecting luminescence at 504˜545 nm can be used to normalize Stokes luminescence image obtained by exciting RhBAM using a laser with a wavelength of 559 nm, and collecting luminescence at 575˜620 nm, thereby obtaining a ratio image reflecting temperature distribution of cytoplasm. Benefits for selecting the excitation and emission wavelengths as said above are that there is almost no interference between the exciting light and emitted light of the two materials, so that two types of exciting lights can be simultaneously used to excite RhBAM and Rh110AM respectively, two emitted fluorescence can be simultaneously collected, there is not time difference between the two collected fluorescent signals, and chronologically, the two collected fluorescent signals can exactly match.

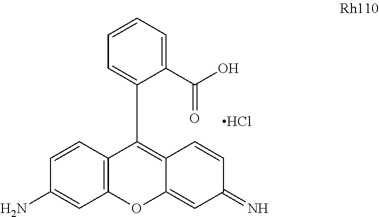 [SMILES: N=c1ccc2c(-c3ccccc3C(=O)O)c3ccc(N)cc3oc-2c1 ; Cl]

Distribution calibration of RhBME (compound of Formula V): Rh800 distributes on mitochondria as RhBNE, therefore, Rh800 can be used in the calibration of RhBME. Stokes luminescence image obtained by exciting Rh800 using a laser with a wavelength of 635 nm, and collecting luminescence at 655˜755 nm can be used to normalize Stokes luminescence image obtained by exciting RhBME using a laser with a wavelength of 559 nm, and collecting luminescence at 575˜620 nm, thereby obtaining a ratio image reflecting temperature distribution of mitochondria. Similar to situations of RhBAM, benefits can be achieved by selecting the wavelengths as said above, that is, there is no interference between the exciting light and emitted light, two types of materials can be simultaneously excited, fluorescence signals can be simultaneously collected, and there is not time difference between the two collected fluorescent signals.

Distribution calibration of RhB-C16 (compound of Formula VI): Rh110-C16 (compound of Formula XI) distributes on cell membrane as RhB-C16, therefore, Rh110-C16 can be used in the calibration of RhB-C16. Stokes luminescence image obtained by exciting Rh110-C16 using a laser with a wavelength of 488 nm, and collecting luminescence at 505˜545 nm can be used to normalize Stokes luminescence image obtained by exciting RhB-C 16 using a laser with a wavelength of 559 nm, and collecting luminescence at 575˜620 nm, thereby obtaining a ratio image reflecting temperature distribution of mitochondria. Similar to situations of RhBAM, benefits can be achieved by selecting the wavelengths as said above, that is, there is no interference between the exciting light and emitted light, two types of materials can be simultaneously excited, fluorescence signals can be simultaneously collected, and there is not time difference between the two collected fluorescent signals.

### Selection of Fluorescent Compound Used in Calibration

According to the rules of the above experiments, it can be known that when a temperature-sensitive fluorescent compound is used to measure the temperature of a biological sample, a material used for calibrating concentration distribution of the temperature-sensitive fluorescent compound can be selected according to the following principles:

1) the fluorescent compound used in calibration will possess the same concentration distribution and localization in a biological sample as the temperature-sensitive fluorescent compound;

2) Stokes luminescence intensity of the fluorescent compound used in calibration is not sensitive to temperature, or is not sensitive to temperature at least in the spectral region selected for collecting fluorescence signal;

3) exciting lights for exciting the fluorescent compound used in calibration and the temperature-sensitive fluorescent compound are of the same wavelength or different wavelength.

Preferably, the difference between wavelengths is greater than 30 nm; more preferably, greater than 40 nm; more preferably, greater than 50 nm; and most preferably, greater than 60 nm;

4) When the exciting lights for exciting the fluorescent compound used in calibration and the temperature-sensitive fluorescent compound are of the same wavelength, the difference between the wave bands or wavelengths for collecting two fluorescent signals is not less than 5 nm; when the difference between the wavelengths of the exciting lights for exciting the fluorescent compound used in calibration and the temperature-sensitive fluorescent compound is great than 30 nm, the difference between the wave bands or wavelengths for collecting two fluorescent signals is not less than 5 nm, and the difference from the wavelength of the exciting light is not less than 5 nm.

When the above conditions are satisfied, simultaneous excitation of the fluorescent compound used in calibration and the temperature-sensitive fluorescent compound and simultaneous collection of produced fluorescence signals can be achieved, and there is no obvious mutual interference between exciting light and fluorescent signals, or between two fluorescent signals, so that chronologically, the two fluorescent signals exactly match, and there is no time difference.

### Methods for Measuring Temperature Distribution Inside Living Cells

Baed on the provided compound of Formula I, a method for measuring temperature distribution inside living cells provided in the present invention, the method comprising:

When anti-Stokes luminescence of a fluorescent compound is used to measure the temperature distribution inside living cells,

(1) the compounds of Formula I is used for staining living cells;

(2) the cells stained in step (1) are imaged under a fluorescence microscope;

(3) fluorescence image is calculated by using Equation (1):

Relative fluorescence intensity:

$$RI = A {e}^{- \frac{Δ E}{{k}_{B} T}} \qquad Equation ( 1 )$$

wherein k_{B} is Boltzmann's constant, T is the absolute temperature, ΔE is activation energy, A is fitting constants, the relative fluorescence intensity is a ratio obtained from anti-Stokes luminescence of the temperature-sensitive fluorescent compound normalized over Stokes luminescence of the compound itself; standard curve of relative fluorescence intensity vs the temperature is pre-determined and Equation (1) is used for calculation, thereby obtaining the distribution image of temperature in living cells;

or

when Stokes luminescence of a fluorescent compound is used to measure the temperature distribution within living cells:

(1) living cells are simultaneously stained by the compound of formula I and a fluorescent compound used in calibration;

(2) the cells stained in step (1) are imaged under a fluorescence microscope;

(3) according to the linear relationship between the change in temperature and relative fluorescence intensity, a pre-determined standard curve is used for calculation to obtain a distribution image of temperature within living cells, wherein relative fluorescence intensity refers to a ratio obtained from Stokes luminescence intensity of the temperature-sensitive fluorescent compound normalized over Stokes luminescence intensity of the fluorescent compound used in calibration.

In a specific embodiment, the compound of formula II , III , IV, V, VI, VII or VIII is used to measure the temperature distribution in living cells.

In a preferred embodiment, the temperature distribution in living cells is the temperature distribution in a subcellular structure; and preferably, the subcellular structure is cell membrane, cytoplasm, or mitochondria.

In a specific embodiment, the compound of formula II or formula III is used in the method for measuring temperature distribution inside living cells of the present invention to measure the temperature distribution of cytoplasm inside living cells. In another specific embodiment, the compound of formula IV or formula V is used in the method for measuring temperature distribution inside living cells of the present invention to measure the temperature distribution of mitochondria inside living cells. In another specific embodiment, the compound of formula VI is used in the method for measuring temperature distribution inside living cells of the present invention to measure the temperature distribution of cell membrane of living cells. In another specific embodiment, the compound of formula VII or VIII is used to measure the temperature of mitochondria inside living cells. In another specific embodiment, the compound of Formula II, Formula X, Formula XI or Formula 2 according to the present invention is used as the fluorescent compound for calibration.

During experiments, the inventors found that a fluorescent dye compound inside a cell will be discharged outside the cell over time, and adding into experimental system organic ion transporter inhibitors can inhibit such process, so that the fluorescence dye compound of the present invention can be maintained wintin a cell or on the cell membrane for a longer time, thereby providing favorable conditions to experiments for measuring temperature inside cells over a longer period.

Accordingly, in a further preferred embodiment, the method for measuring temperature distribution inside living cells according to the present invention further comprises using organic ion transporter inhibitors to inhibit the fluorescent dye compound from being discharged outside the cells, while measuring the temperature at the same time. In a specific embodiment, the organic ion transporter inhibitor is probenecid, sulfinpyrazone or MK571.

### Kits for Measuring Temperature Distribution Inside Living Cells

Baed on the provided compound of Formula I and uses thereof, a kit for measuring temperature distribution inside living cells is further provided in the present invention, said kit comprising:

(1) the compound of formula I according to the present invention;

(2) auxiliary reagents used in cell staining (e.g., DMSO, which is a cosolvent, and 50000 times of stock solution of dye (10 mM) can be formulated by using DMSO, and stored at −20° C., and then diluted in an extracellular solution used in the experiment, such as PBS , Tyrode solution etc., to final concentration);

(3) container accommodating the compounds and auxiliary reagents as mentioned above; and

(4) an instruction manual for measuring temperature distribution inside living cells using the compound.

In a specific embodiment, the compound is the compound of formula II , III , IV, V, VI, VII or VIII.

In a preferred embodiment, the compound of formula II or formula III is used to measure the temperature distribution of cytoplasm inside living cells for measuring temperature distribution inside living cells.

In another preferred embodiment, the compound of formula IV or formula V is used to measure the temperature distribution of mitochondria inside living cells for measuring temperature distribution inside living cells.

In another preferred embodiment, the compound of formula VI is used to measure the temperature distribution of cell membrane of living cells for measuring temperature distribution inside living cells.

In another preferred embodiment, the compound of formula VII or VIII is used to measure the temperature of mitochondria inside living cells.

In a further embodiment, the kit further comprises the compound of Formula X, Formula XI or Formula 2.

### Advantages of the Invention

1. The fluorescent dye compounds of the present invention are capable of staining subcellular structures of a living cell, especially the cell membrane, cytoplasm, or mitochondria, thereby obtaining temperature distribution image of the cell with high spatial and temporal resolution;

2. The present invention provides a powerful tool for studying cell metabolism, cell inflammatory fever and the like;

3. The present invention provides a novel cellular thermal imaging method and a powerful tool for observing changes in the cellular temperature when the cell is subjected to various processes and pathological conditions;

4. It is a creative use of another referential fluorescent compound in distribution calibration of a temperature-sensitive fluorescent compound, wherein said another fluorescent compound possesses the same intracellular concentration distribution as the temperature-sensitive fluorescent compound and does not possess temperature-sensitive properties, thereby more accurately measuring the intracellular temperature;

5. The method of the present invention can be easily applied in a variety of fluorescence microscopy imaging system, and accurately, easily and rapidly obtain temperature distribution image of a cell with high spatial and temporal resolution, therefore, the method can be simply expanded and applied.

The invention will be further illustrated with reference to the following specific examples. It is to be understood that these examples are only intended to illustrate the invention, but not to limit the scope of the invention. For the experimental methods in the following examples without particular conditions, they are performed under routine conditions, or as instructed by the manufacturer. All the percentages or fractions refer to weight percentage and weight fraction, unless stated otherwise.

Unless otherwise defined, all the technical and scientific terms used in the present specification have the meanings as commonly understood by those skilled in the art. In addition, any method and material which are similar or equivalent with the contents disclosed herein can be applied in the present methods. The preferred methods and materials for carrying out the present invention described herein are only given as examples.

### EXAMPLE 1

### Synthesis of Rh101AM and RhBAM

Rh101 (purchased from Santa Cruz), cesium fluoride, and bromoacetic acid were mixed at a ratio of 1:2:1.2 and dissolved in ten times of dimethylformamide (DMF). The reaction mixture was stirred for 2 hours at room temperature, and then purified with preparative high performance liquid chromatography to obtain Rh101AM (the compound of Formula II).

Synthesis method for RhBAM is similar to that of Rh101AM:

RhB (purchased from Santa Cruz), cesium fluoride, and bromoacetic acid were mixed at a ratio of 1:2:1.2 and dissolved in ten times of dimethylformamide (DMF). The reaction mixture was stirred for 2 hours at room temperature, and then purified with preparative high performance liquid chromatography to obtain RhBAM (the compound of Formula III).

### EXAMPLE 2

### Synthesis of Rh101ME and RhBME

Rh101 and thionyl chloride were mixed at a ratio of 1:5 and dissolved in 10 times of chloroform, heated to 60° C. and stirred for 10 minutes. And then the mixture was cooled to room temperature and quenched with methanol. Afterwards, the solvent was removed on a rotary evaporator under reduced pressure, and the residue was purified through preparative high performance liquid chromatography thereby obtaining Rh101ME (the compound of Formula IV).

Synthesis method for RhBME is similar to that of Rh101ME:

RhB and thionyl chloride were mixed at a ratio of 1:5 and dissolved in 10 times of chloroform, heated to 60° C. and stirred for 10 minutes. And then the mixture was cooled to room temperature and quenched with methanol. Afterwards, the solvent was removed on a rotary evaporator under reduced pressure, and the residue was purified through preparative high performance liquid chromatography thereby obtaining RhBME (the compound of Formula V).

### EXAMPLE 3

### Measurement of the Temperature Distribution of Cytoplasm Using Rh101AM

Rh101AM was used to stain live cells and imaged under a fluorescent microscope. The fluorescenc image was calculated using equation (1), thereby obtaining the image of intracellular temperature distribution.

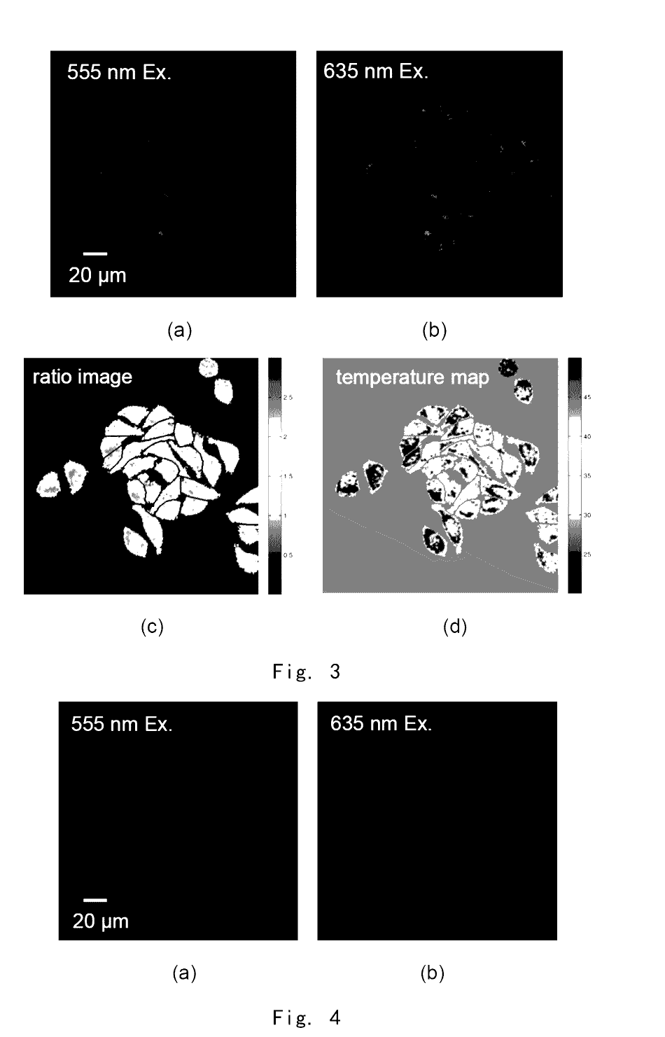

FIG. 3 shows Stokes luminescence image of HepG2 cells captured by EMCCD (Evolve 512, Photometrice Ltd.) under fluorescence microscope (BX61WI, Olympus Ltd., 40×lens, numerical aperture 0.8, the temperature of culture solution is 27.9□ at imaging), after the cells were stained by using 200 nM Rh101AM for 60 mins in an incubator at 37□. Wherein, 3(a) shows Stokes luminescence image from a monochromator (Optoscan monochromator, Cairn Research Ltd.), excited at a wavelength of 555 nm (bandwidth of 3 nm) and collected at 573˜613 nm; 3(b) shows anti-Stokes luminescence image from a monochromator, excited at a wavelength of 635 nm (bandwidth of 15 nm) and collected at 573˜613 nm; and 3(c) shows a ratio image obtained from normalizing FIG. 3(b) over FIGS. 3(a); and 3(d) shows the temperature distribution of cells calculated according to equation (1).

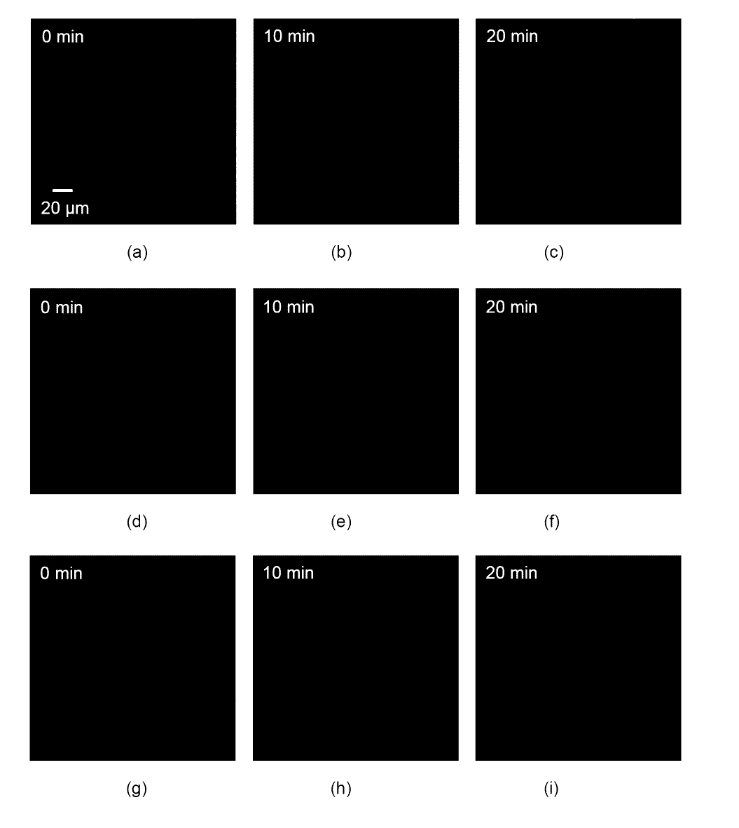

The results showed that the temperature is not uniform as generally assumed. Kachynski et al., have used Rh101 for measuring temperature inside a living cell, and displayed that the temperature inside a living cell is uniform [4], which is inconsistent with the results of the present invention. In order to explore underlying reasons, Rh101 was used by the present inventors to stain living cells under the same conditions as aforementioned Rh101AM, images were obtained, and obtained Stokes luminescence image and anti-Stokes luminescence image are shown in FIG. 4. The results show that clarity of Stokes luminescence image and anti-Stokes luminescence image obtained by staining living cells with Rh101 is much poor compared with that of Rh101AM, and there is no spatially specific distribution. What is more important is that, compared with FIG. 3(a), the fluorescence intensity in FIG. 4(a) is not significantly reduced, indicating that, in both figures, the concentration of Rh101 is similar. However, compared with FIG. 3(b), anti-Stokes luminescence of Rh101 shown in FIG. 4(b) is much weaker. Since anti-Stokes luminescence positively correlates with temperature, this result reflects that the temperature of cells shown in FIG. 4 is much lower than that shown in FIG. 3. However, the imaging conditions and temperature conditions are identical between the two figures, therefore, the temperature level of cells shown in FIG. 4 is not correct.

Cell membrane itself is a good heat-insulating material, and metabolic activities of living cells can also produce some heat to maintain the temperature of cells, therefore, intracellular temperature won't immediately decrease when cells are changed from culture conditions at 37□ to imaging conditions at 28□. The reason why the temperature of cell reflected in FIG. 4 is lower is probably that Rh101 does not enter the cell, but merely adheres outside the cell (the fluorescence intensity shown in FIG. 4(a) is not significantly lower that that shown in FIG. 3(a)), therefore cells were subject to a lower temperature of solution, and anti-Stokes luminescence of Rh101 shown in FIG. 4(b) is significantly weaker than that shown in FIG. 3(b).

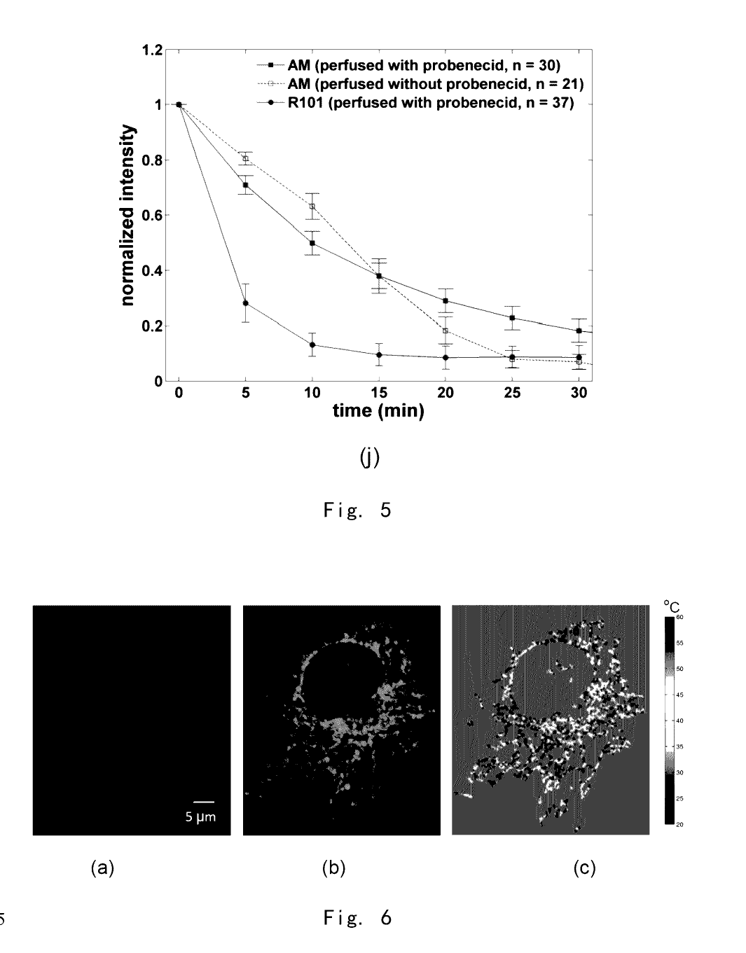

Compared with the dye entering into the cell, the dye adhering outside the cell will be easily eluted. To further demonstrate that Rh101 was adhered outside the cell, after HepG2 cells were stained under the same dyeing conditions as said described, staining cells with the dye under slightly stronger elution conditions was observed by a perfusion method (results shown in FIG. 5). Wherein FIGS. 5(a-c) show Stokes luminescence image from a monochromator, excited at a wavelength of 555 nm (bandwidth of 3 nm) and collected at 573˜613 nm, respectively, after cells were stained with Rh101AM and lavaged with 2.5 mM probenecid perfusion solution (Tyrode solution) for 0 min, 10 min, 20 min. FIGS. 5(d-f) show Stokes luminescence image from a monochromator, excited at a wavelength of 555 nm (bandwidth of 3 nm) and collected at 573˜613 nm, respectively, after cells were stained with Rh101AM and lavaged with perfusion solution without probenecid (Tyrode solution) for 0 min, 10 min, 20 min. FIGS. 5(g-i) show Stokes luminescence image from a monochromator, excited at a wavelength of 555 nm (bandwidth of 3 nm) and collected at 573˜613 nm, respectively, after cells were stained with Rh101 and lavaged with 2.5 mM probenecid perfusion solution

(Tyrode solution) for 0 min, 10 min, 20 min. FIG. 5(j) shows curves of Stokes luminescence intensity vs perfusion time under the above 3 conditions. Wherein the function of probenecid is to inhibit organic ion transporters present on cell membrane, and the function of organic ion transporters is to transport dyes entering into cells outside the cells. The results showed that after the cells were stained with Rh101 and perfused with a perfusion solution containing probenecid for 10 mins, Stokes luminescence of the cells almost disappeared (FIG. 5(h, j)); and after the celle were stained with Rh101AM and perfused with a perfusion solution containing or not containing probenecid for 10 mins, there was a considerable amount of fluorescent maintained in cells (50% or higher, FIG. 5(b, e, j)). This result suggests that most of Rh101 binds to cell surface and can be easily eluted, while Rh101AM is significantly and intracellularly enriched and is difficult to be eluted. Considering the fact that the temperature of a cell (the temperature reflected by anti-Stokes luminescence) obtained from Rh101 staining is lower than that obtained from Rh101AM staining, it can be known that most of Rh101 merely adheres outside the cell, instead of entering into the cell, the obtain image of temperature only reflects the temperature of the cell surface, it is difficult to reflect intracellular temperature distribution, and the image of temperature distribution obtained using Rh101AM staining will truly reflect the distribution of intracellular temperature. Furthermore, the present inventors have also discovered that it is also difficult for RhB to enter a cell.

Additionally, the results show that after cells were stained with Rh101AM, the fluorescence intensity of the cells will tend to be reduced with prolonged perfusion time, and after the cells were perfused with a perfusion solution not containing probenecid for 25 mins, Stokes luminescence of the cells almost disappeared (FIG. 5(j)), however, after the cells were perfused with a perfusion solution containing probenecid over 30 mins, there was still certain Stokes luminescence (FIG. 5(j)). The results indicate that most of the dyes will be eluted due to exocytosis over a longer elution time, however, Rh101AM leakage can be reduced by inhibiting the organic ion transporter using probenecid, which provides favorable conditions for experiments, for which it is necessary to determine the temperature of a cell over a longer period.

### EXAMPLE 4

### Measurement of the Temperature Distribution of Mitochondria Using Rh101ME

Rh101ME and Rh800 (the compound of formula 2) were used to stain live cells and imaged under a confocal laser fluorescence microscope. The fluorescence image was calculated using equation (1), thereby obtaining the image of temperature distribution of mitochondria.

FIG. 6 shows image of COS7 cells under laser confocal fluorescence microscope (FV1000, Olympus, 60× water immersed lens, numerical aperture 1.2, the temperature of culture solution is 30° C. at imaging), after the cells were stained using 100 nM Rh101ME and 100 nM Rh800 for 30 mins in an incubator at 37□. Wherein FIG. 6(a) shows Stokes luminescence image produced by Rh800 excited by a laser at 635 nm and collected at 655˜755 nm; FIG. 6(b) shows anti-Stokes luminescence image produced by Rh101ME excited by a laser at 635 nm and collected at 575˜620 nm; and FIG. 6(c) shows temperature distribution image of mitochondria obtained through calculation according to equation (1), after normalizing FIG. 6(b) over FIG. 6(a). The results show that there is a difference in the temperature within mitochondria.

### EXAMPLE 5

### Measurement of the Temperature Distribution of Mitochondria Using RhBME

RhBME and Rh800 were used to stain live cells and imaged under a confocal laser fluorescence microscope. The image of temperature distribution of mitochondria was obtained by calculating the ratio image using a standard curve according to the linear relationship of relative Stokes luminescence intensity vs temperature.

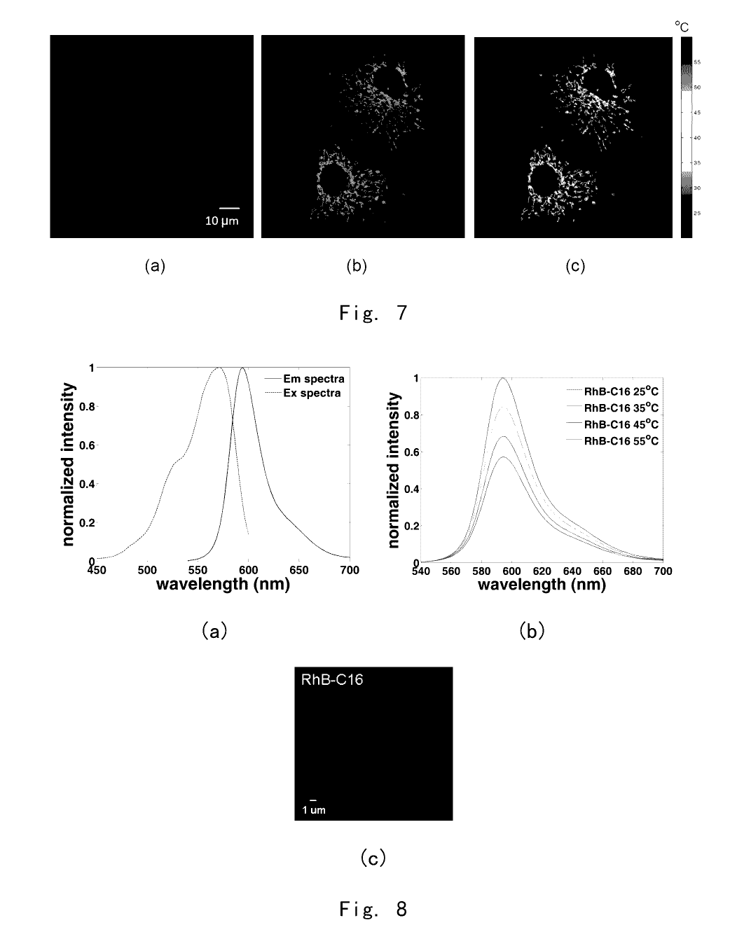

FIG. 7 shows image of COST cells under laser confocal fluorescence microscope (FV1000, Olympus, 60× water immersed lens, numerical aperture 1.2, the temperature of culture solution is 30° C. at imaging), after the cells were stained by using 50 nM RhBME and 50 nM Rh800 for 30 mins in an incubator at 37□. Wherein FIG. 7(a) shows Stokes luminescence image produced by RhBME excited by a laser at 559 nm and collected at 575˜620 nm; FIG. 7(b) shows Stokes luminescence image produced by Rh800 excited by a laser at 635 nm and collected at 655˜755 nm; and a ratio image reflecting temperature distribution of mitochondria was obtained by normalizing FIG. 7(b) over FIG. 7(a). The image of temperature distribution of mitochondria as shown in FIG. 7(c) was obtained by calculation according to the linear relationship of relative Stokes luminescence intensity vs temperature.

The results show that there is a difference in the temperature within mitochondria, which is consistent with the detection results obtained from Rh101ME.

### EXAMPLE 6

### Measurement of the Temperature Distribution of Cytoplasm Using RhBAM

Example 5 was repeated, except that RhBAM and Rh110AM (the compound of formula X), instead of RhBME and Rh800 were used to stain live cells and imaged under a confocal laser fluorescence microscope. Stokes luminescence image obtained by exciting Rh110AM using a laser with a wavelength of 488 nm, and collecting luminescence at 504˜545 nm can be used to normalize Stokes luminescence image obtained by exciting RhBAM using a laser with a wavelength of 559 nm, and collecting luminescence at 575˜620 nm, thereby obtaining a ratio image reflecting temperature distribution of cytoplasm. The image of temperature distribution of cytoplasm was obtained by calculation according to the linear relationship of relative fluorescence intensity vs temperature. The results also showed that the temperature distribution within a cell is not uniform (measurement results not shown).

### EXAMPLE 7

### Synthesis of RhB-C16 (the Compound of Formula VI)

7 g of rhodamine B was suspended in 10 ml of dry benzene, and 3 ml of dry pyridine was added and homogeneously mixing. 27 ml of thionyl chloride was added dropwise with stirring and cooling. At room temperature, the reaction mixture was stirred for 12 hours. Then 1 g of cetyl alcohol was added, and stirred for another 12 hours. Benzene was removed by evaporation, the obtained powder was dissolved in a small amount of ethanol, and the resulting solution was spotted on chromatographic plate and then developped in a solvent system (petroleum ether and ethyl acetate), and then developped in diethyl ether to remove the cetyl alcohol in the product. The product was resuspended in ethanol, and separated through chromatography for two times. The final ethanol solution was evaporated to give the product as a waxy solid.

### EXAMPLE 8

### Measurement of the Temperature Distribution of Cytoplasm Using RhB-C16

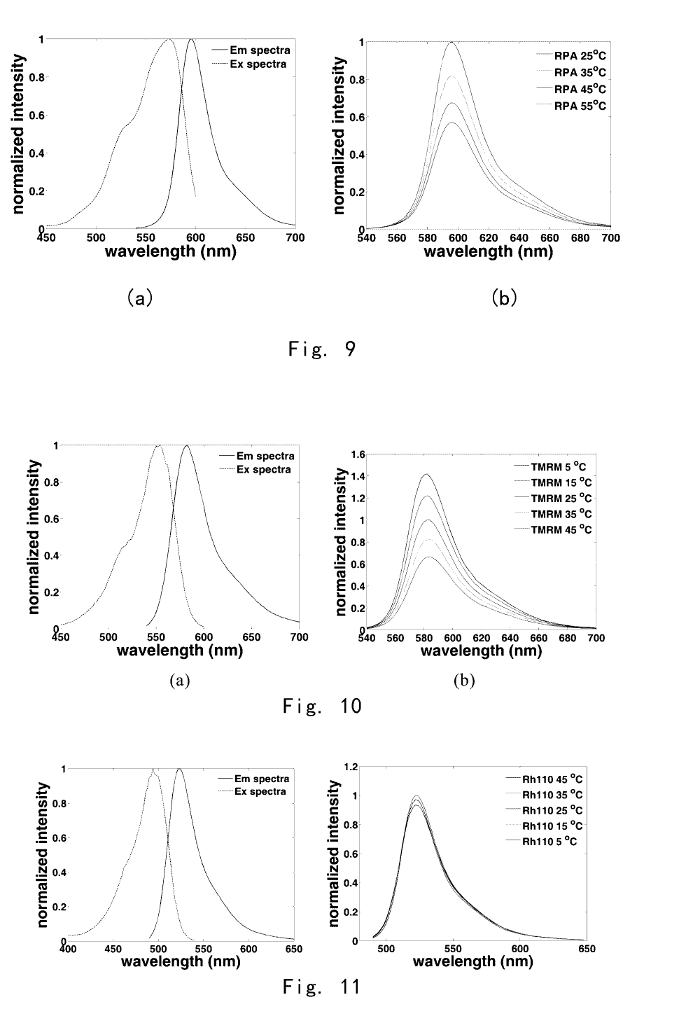

FIG. 8(a) shows spectral properties of RhB-C16; FIG. 8(b) shows that Stokes luminescence of RhB-C16 possesses temperature-sensitive properties as other derivatives of RhB; and FIG. 8(c) shows image of cells under fluorescence microscope, after the cells were stained by using RhB-C 16, thereby demonstrating that RhB-C16 is clearly positioned on cell membrane. Therefore, RhB-C16 can be used to determine temperature distribution of cell membrane.

Example 5 was repeated, except that RhB-C16 and Rh110-C16 (the compound of formula XI), instead of RhBME and Rh800 were used to stain live cells and imaged under a confocal laser fluorescence microscope. Stokes luminescence image obtained by exciting Rh110-C16 using a laser with a wavelength of 488 nm, and collecting luminescence at 504˜545 nm can be used to normalize Stokes luminescence image obtained by exciting RhB-C16 using a laser with a wavelength of 559 nm, and collecting luminescence at 575˜620 nm, thereby obtaining a ratio image reflecting temperature distribution of cytoplasm. The image of temperature distribution of cytoplasm was obtained by calculation according to the linear relationship of relative fluorescence intensity vs temperature.

### EXAMPLE 9

### Temperature-Sensitive Properties of Other Compounds

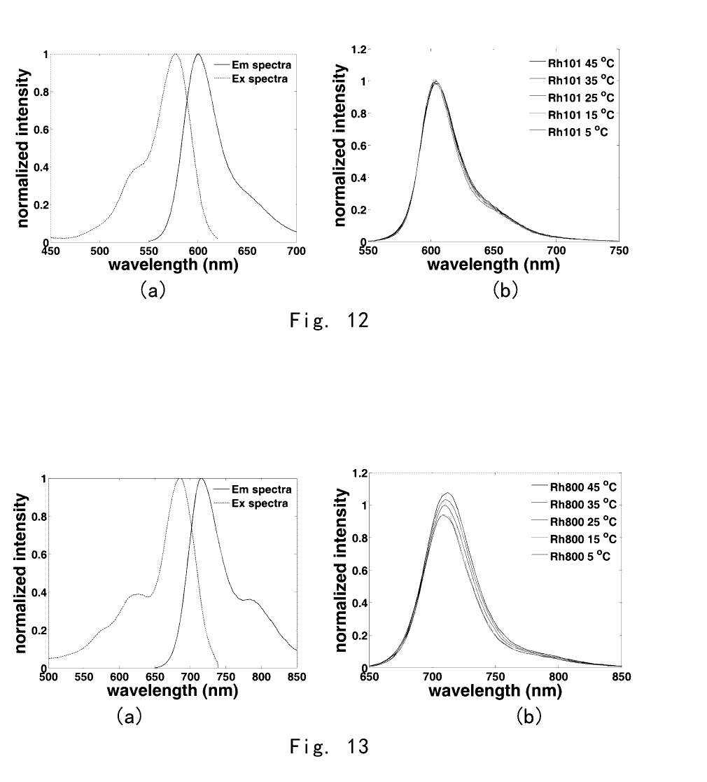

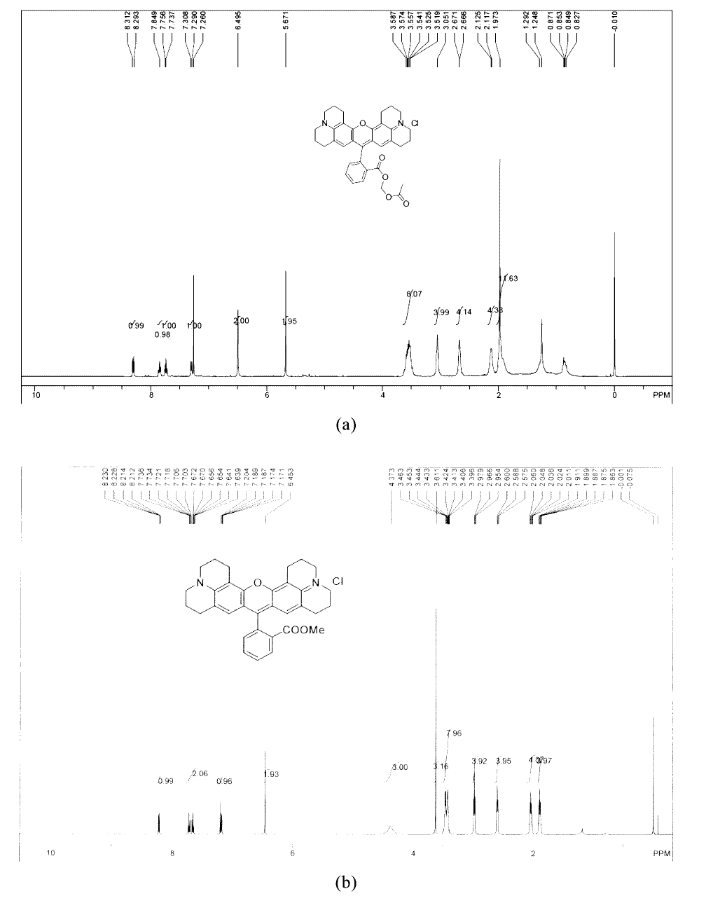

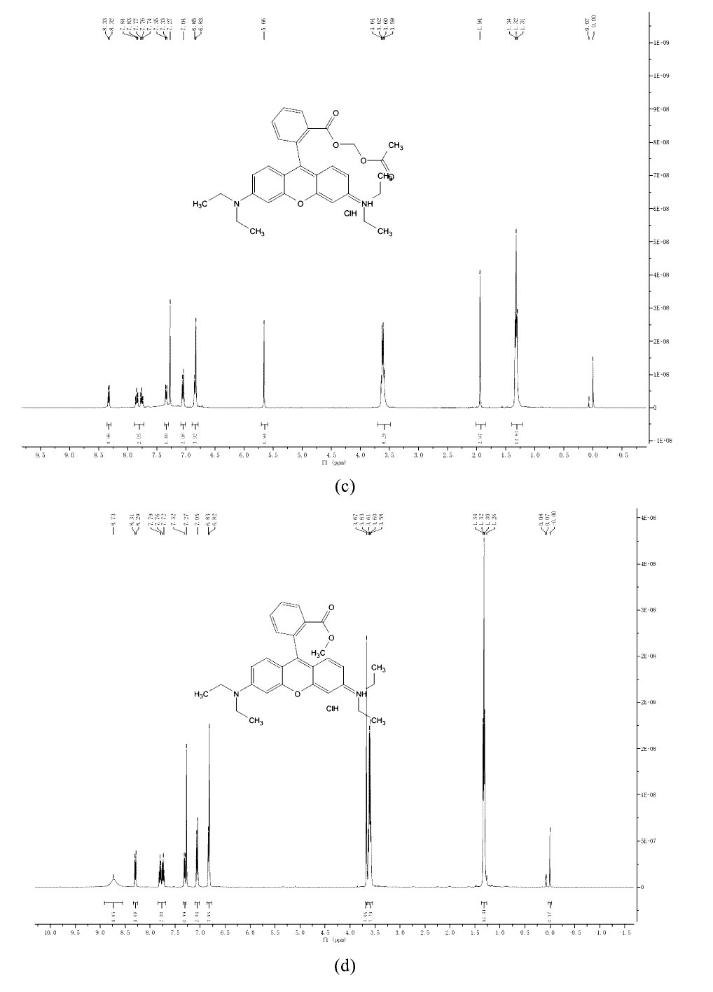

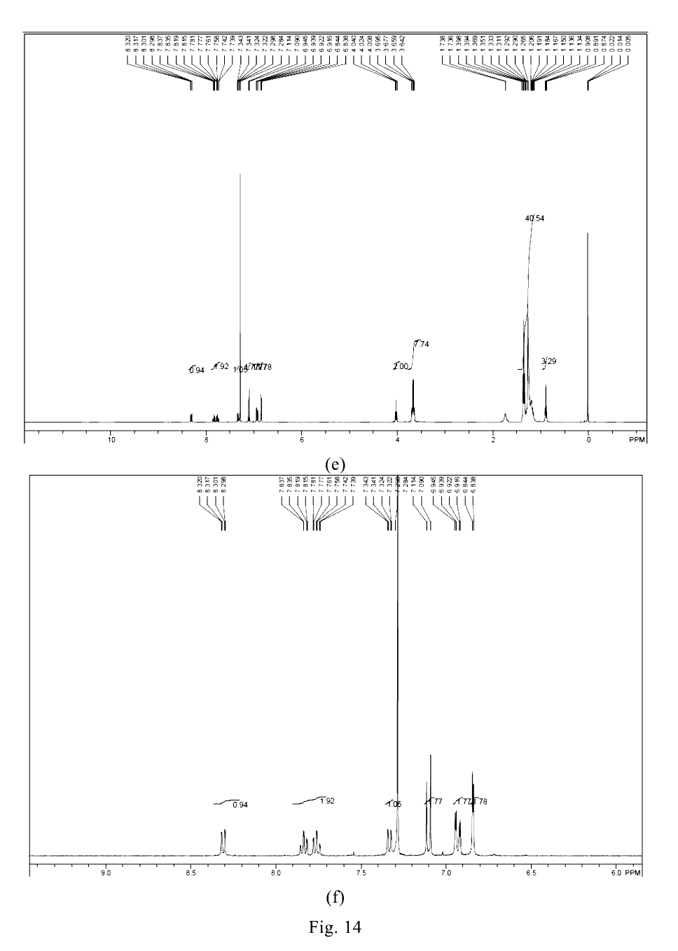

The inventors have further tested spectral properties and temperature-sensitive properties of the compound of formula VII (Rhodamine B-[(1,10-phenanthrolin-5-yl) aminocarbonyl]benzyl ester, abbreviated as RPA), the compound of Formula VIII (tetramethylrhodamine methyl ester , abbreviated as TMRM) as well as Rh110 compound, Rh101 compound and Rh800 compound, and discover that Stokes luminescence of the compound of formula VII and the compound of formula VIII possess temperature-sensitive properties (see FIGS. 9 and 10); Stokes luminescence of Rh110 compound and Rh101 compound does not possess temperature-sensitive properties (see FIG. 11-12); and Stokes luminescence of Rh800 compound does not possess temperature-sensitive properties at a wavelength of less than 700 nm (see FIG. 13). RPA and TMRM are fluorescent dyes known as being localized in mitochondria, so that they can be used to determine the temperature distribution of mitochondria within living cells.

### EXAMPLE 10

### Synthesis of the Compounds of Formula X and XI

Rh110 (purchased from Santa Cruz), cesium fluoride, and bromoacetic acid were mixed at a ratio of 1:2:1.2 and dissolved in ten times of dimethylformamide (DMF). The reaction mixture was stirred for 2 hours at room temperature, and then purified with preparative high performance liquid chromatography to obtain the compound of Formula X, Rh110AM.

7 g of Rh110 was suspended in 10 ml of dry benzene, and 3 ml of dry pyridine was added and homogeneously mixing. 27 ml of thionyl chloride was added dropwise with stirring and cooling. At room temperature, the reaction mixture was stirred for 12 hours. Then 1 g of cetyl alcohol was added, and stirred for another 12 hours. Benzene was removed by evaporation, the obtained powder was dissolved in a small amount of ethanol, and the resulting solution was spotted on chromatographic plate and then developped in a solvent system (petroleum ether and ethyl acetate), and then developped in diethyl ether to remove the cetyl alcohol in the product. The product was resuspended in ethanol, and separated through chromatography for two times. The final ethanol solution was evaporated to give the product, Rh110-C16 (the compound of formula XI).

### DISCUSSION

The temperature distribution of cell (FIG. 3(d)) and the temperature distribution of mitochondria (FIG. 6(c), FIG. 7(c)) obtained by using the method of the present invention show that the temperature within a cell or mitochondria is not uniform as generally assumed. However, in the prior art, for example reportes from Kachynski et al., indicate that there is no significant fluctuation in the intracellular temperature distribution [4], because the used dye itself is difficult to penetrate cell membrane, however, the literature did not notice or suggested the presence of such problem or defect, therefore, the obtained results are not reliable.

Other programs of temperature measurement in the prior art, for example, a method using a thermocouple to measure temperature of a single location in a cell possesses the merit of high temporal resolution, however, when it is applied to the measurement of two-dimensional temperature distribution in a cell, the temporal resolution will be greatly reduced. Additionally, for such contact-type measurement of temperature based on thermocouple probe, cells are likely to be damaged during the two-dimensional scanning process. The method of the present invention will not damage cells, and possess sufficient temporal resolution. For the method using Stokes luminescence of a fluorescent compound itself in calibration, the time interval will be a few second, or even less than one second (depending on imaging speed); while for the method using another fluorescent compound in calibration, there is no time difference.

When hydrophilic temperature-sensitive fluorescent nano material in the prior art is used as a material for measuring temperature in a cell, using average fluorescence intensity of the whole cell to reflect the temperature of the cell will be inacurate due to the uneven aggregation of the material [1]. The temperature-sensitive fluorescent dye compound of the present invention can enter into a cell, and a temperature distribution image can be obtained with high spatial resolution, thereby distinguishing the temperature at different locations within a cell. In summary, the method of the present invention meet the requirements on small size and rapid measurement for measuring intracellular temperature, thereby achieving a high spatial and temporal-resolution. Compared with the prior art, the method of the present invention has a significant advantage in the measurement of temperature in a cell.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

### REFERENCES

1. Gota, C., et al., Hydrophilic Fluorescent Nanogel Thermometer for Intracellular Thermometry. Journal of the American Chemical Society, 2009. 131(8): p. 2766-+.

2. Clark, J. L. and G. Rumbles, Laser cooling in the condensed phase by frequency up-conversion. Phys Rev Left, 1996. 76(12): p. 2037-2040.

3. Clark, J. L., P. F. Miller, and G. Rumbles, Red edge photophysics of ethanolic rhodamine 101 and the observation of laser cooling in the condensed phase. Journal of Physical Chemistry A, 1998. 102(24): p. 4428-4437.

4. Kachynski, A. V., et al., Three-dimensional confocal thermal imaging using anti-Stokes luminescence. Applied Physics Letters, 2005. 87(2).

5. Chen, Y. Y. and A. W. Wood, Application of a Temperature-Dependent Fluorescent Dye (Rhodamine B) to the Measurement of Radiofrequency Radiation-Induced Temperature Changes in Biological Samples. Bioelectromagnetics, 2009. 30(7): p. 583-590.

1. A compound of formula I, 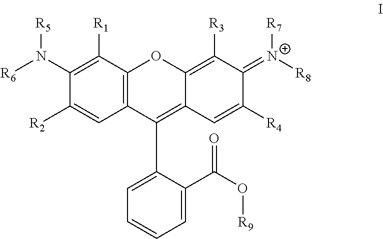
wherein,
R₉ is a hydrocarbyl of 1-22 carbon atoms, or an alkyl of 1 to 3 carbon atoms substituted with an ester group of 2-3 carbon atoms,
all of R₅, R₆, R₇, R₈ are a hydrocarbyl or H, and
all of R₁, R₂, R₃, R₄ are H or a lower hydrocarbyl;
or
R₉ is a hydrocarbyl of 2-22 carbon atoms, or an alkyl of 1 to 3 carbon atoms substituted with an ester group of 2-3 carbon atoms, and
R₅ and R₁, R₆ and R₂, R₇ and R₃, R₈ and R₄ are connected into a six-membered ring.

2. The compound of claim 1, wherein the compound is a compound of following formula:

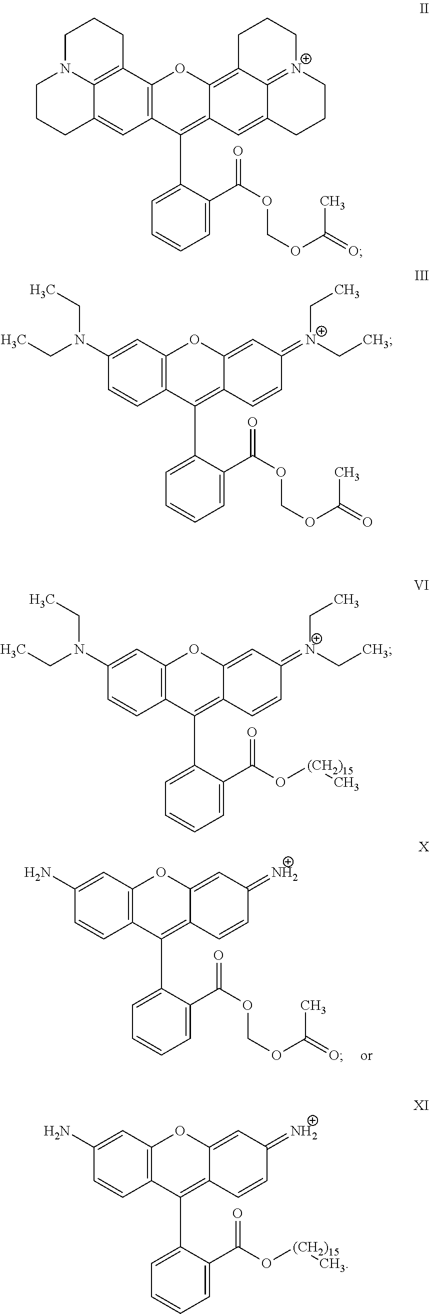

3. Use of a compound of formula I in the measurement of temperature distribution in a living cell. 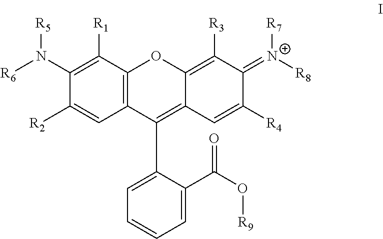
wherein,
R₉ is selected from the group consisting of a hydrocarbyl of 1-22 carbon atoms, or an alkyl of 1 to 3 carbon atoms substituted with an ester group of 2-3 carbon atoms, and an alkyl of 1 to 3 carbon atoms substituted with an aryl;
each of R₅, R₆, R₇, and R₈ is independently selected from a hydrocarbyl, and all of R₁, R₂, R₃, R₄ are H or a lower hydrocarbyl;
or
R₉ is selected from the group consisting of a hydrocarbyl of 1-22 carbon atoms, or an alkyl of 1 to 3 carbon atoms substituted with an ester group of 2-3 carbon atoms, and an alkyl of 1 to 3 carbon atoms substituted with an aryl; and
R₅ and R₁, R₆ and R₂, R₇ and R₃, R₈ and R₄ are connected into a six-membered ring.

4. The use of claim 3, wherein the compound is a compound of the following formula:

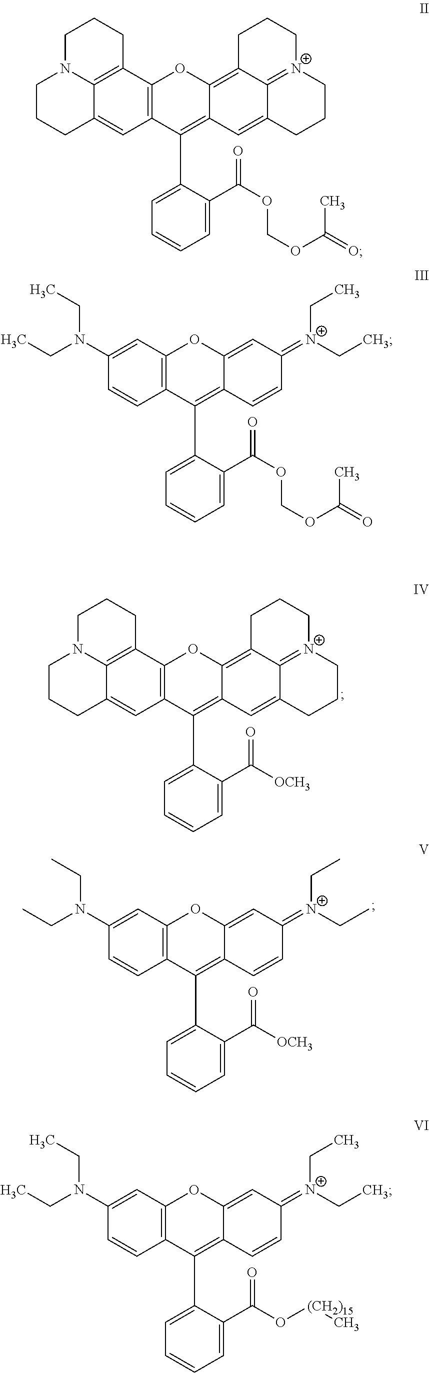

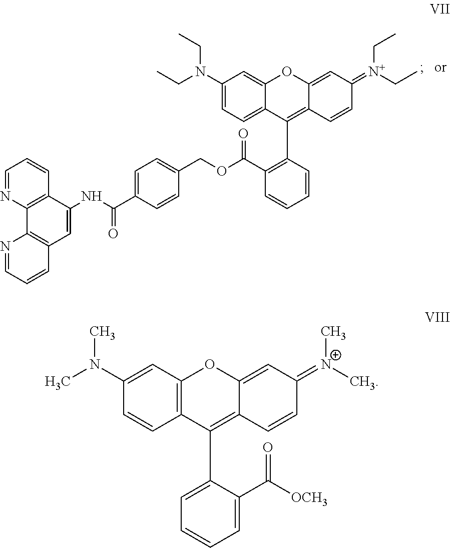

5. The use of claim 3, wherein the temperature distribution within a living cell is the temperature distribution in a subcellular structure; preferably, the subcellular structure is cell membrane, cytoplasm, or mitochondria.

6. Use of a compound of Formula I or Formula 2 in the calibration of distribution of a temperature-sensitive fluorescent compound when the temperature-sensitive fluorescent compound is used to measure the temperature distribution in a living cell,
wherein, 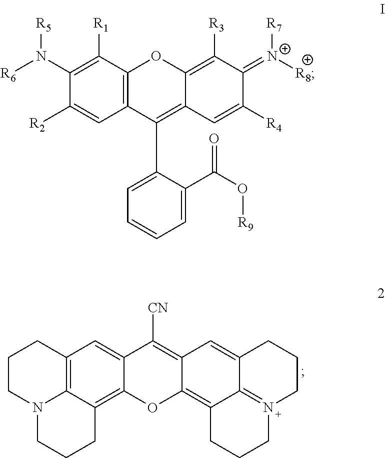
R₉ is a hydrocarbyl of 1-22 carbon atoms, or an alkyl of 1 to 3 carbon atoms substituted with an ester group of 2-3 carbon atoms,
all of R₅, R₆, R₇, and R₈ are H, and
all of R₁, R₂, R₃, R₄ are H or a lower hydrocarbyl;
or
R₉ is a hydrocarbyl of 1-22 carbon atoms, or an alkyl of 1 to 3 carbon atoms substituted with an ester group of 2-3 carbon atoms, and
R₅ and R₁, R₆ and R₂, R₇ and R₃, R₈ and R₄ are connected into a six-membered ring.

7. The use of claim 6, wherein the compound is a compound of the following formula:

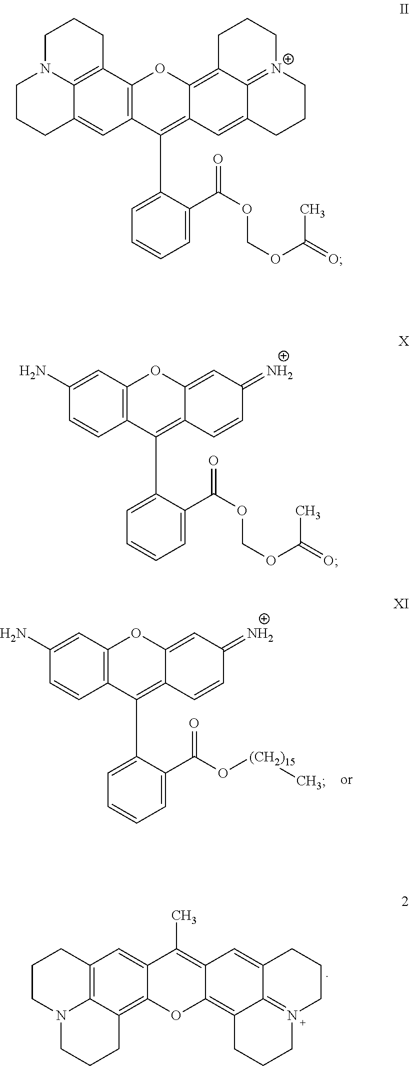

8. A method for measuring temperature distribution in a living cell, said method including the following steps:
when anti-Stokes luminescence imaging of a temperature-sensitive fluorescent compound is used to measure the temperature distribution in a living cell,
(1) a compound of Formula I is used for staining living cells; 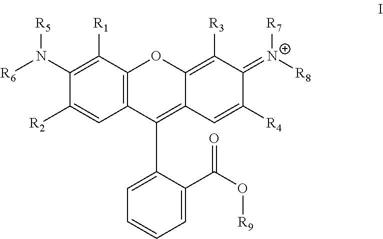
wherein,
R₉ is selected from the group consisting of a hydrocarbyl of 1-22 carbon atoms, an alkyl of 1 to 3 carbon atoms substituted with an ester group of 2-3 carbon atoms and an alkyl of 1 to 3 carbon atoms substituted with an aryl,
R₅, R₆, R₇, R₈ are independently selected from a hydrocarbyl, and
all of R₁, R₂, R₃, R₄ are H or a lower hydrocarbyl;
or
R₉ is selected from the group consisting of a hydrocarbyl of 1-22 carbon atoms, an alkyl of 1 to 3 carbon atoms substituted with an ester group of 2-3 carbon atoms and an alkyl of 1 to 3 carbon atoms substituted with an aryl, and
R₅ and R₁, R₆ and R₂, R₇ and R₃, R₈ and R₄ are connected into a six-membered ring;
(2) the cells stained in step (1) are imaged under a fluorescence microscope;
(3) fluorescence image is calculated by using Equation (1):
Relative fluorescence intensity:
$$RI = A {e}^{- \frac{Δ E}{{k}_{B} T}} \qquad Equation ( 1 )$$
wherein k_{B} is Boltzmann's constant, T is the absolute temperature, ΔE is activation energy, A is fitting constants, and the relative fluorescence intensity is a ratio obtained from anti-Stokes luminescence of the compound of formula I normalized over Stokes luminescence of the compound itself,
standard curve of relative fluorescence intensity vs the temperature is pre-determined and Equation (1) is used for calculation, thereby obtaining the distribution image of temperature in living cells;
or
when Stokes luminescence or anti-Stokes luminescence imaging of a temperature-sensitive fluorescent compound is used to measure the temperature distribution within living cells,
(1) living cells are stained by a compound of formula I and a fluorescent compound used in calibration; 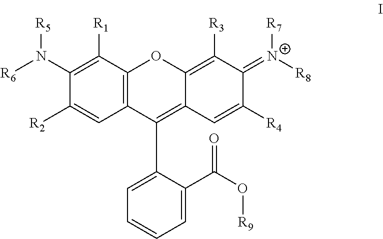
wherein,
R₉ is selected from the group consisting of a hydrocarbyl of 1-22 carbon atoms, an alkyl of 1 to 3 carbon atoms substituted with an ester group of 2-3 carbon atoms and an alkyl of 1 to 3 carbon atoms substituted with an aryl,
R₅, R₆, R₇, R₈ are independently selected from a hydrocarbyl, and
R₁, R₂, R₃, R₄ are H or a lower hydrocarbyl;
or
R₉ is selected from the group consisting of a hydrocarbyl of 1-22 carbon atoms, an alkyl of 1 to 3 carbon atoms substituted with an ester group of 2-3 carbon atoms and an alkyl of 1 to 3 carbon atoms substituted with an aryl, and
R₅ and R₁, R₆ and R₂, R₇ and R₃, R₈ and R₄ are connected into a six-membered ring;
(2) the cells stained in step (1) are imaged under a fluorescence microscope;
(3) according to the linear relationship between the change in temperature and relative fluorescence intensity, a pre-determined standard curve is used for calculation to obtain a distribution image of temperature within living cells, wherein the relative fluorescence intensity refers to a ratio obtained from Stokes or anti-Stokes luminescence intensity of the temperature-sensitive fluorescent compound normalized by Stokes luminescence intensity of the fluorescent compound used in calibration.

9. The method of claim 8, wherein the compound of formula I is the compound of the following formula:

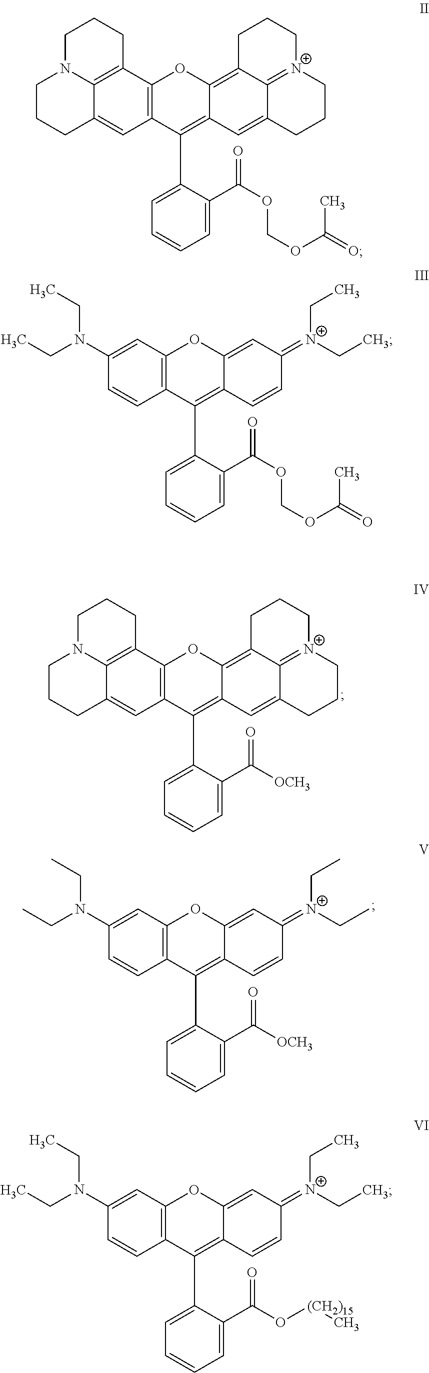

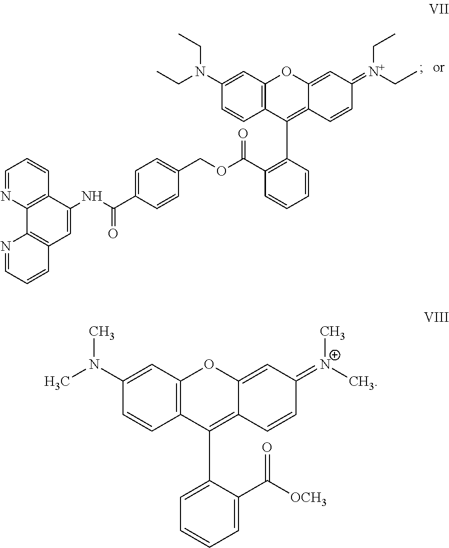

10. The method of claim 8, wherein the temperature distribution within a living cell is the temperature distribution in a subcellular structure; preferably, the subcellular structure is cell membrane, cytoplasm, or mitochondria.

11. A calibration method for the distribution of a temperature-sensitive fluorescent compound when the temperature-sensitive fluorescent compound is used to measure the temperature distribution within a live cell, wherein another fluorescent compound without temperature-sensitive properties is used in the calibration of the distribution of the temperature-sensitive fluorescent compound, and said another fluorescent compound possesses the same intracellular concentration distribution as the temperature-sensitive fluorescent compound.

12. The method of claim 11, wherein said another fluorescent compound without temperature-sensitive properties is covalently connected with said temperature-sensitive fluorescent compound; preferably, said another fluorescent compound without temperature-sensitive properties is covalently connected with said temperature-sensitive fluorescent compound through a hydrocarbon chain; more preferably, said another fluorescent compound without temperature-sensitive properties is covalently connected with said temperature-sensitive fluorescent compound through a hydrocarbon chain of 2-18 carbon atoms; and most preferably, said another fluorescent compound without temperature-sensitive properties is covalently connected with said temperature-sensitive fluorescent compound through a hydrocarbon chain of 4-10 carbon atoms.

13. The method of claim 11, wherein the following compounds are used in the method for the calibration of distribution of the temperature-sensitive fluorescent compound:

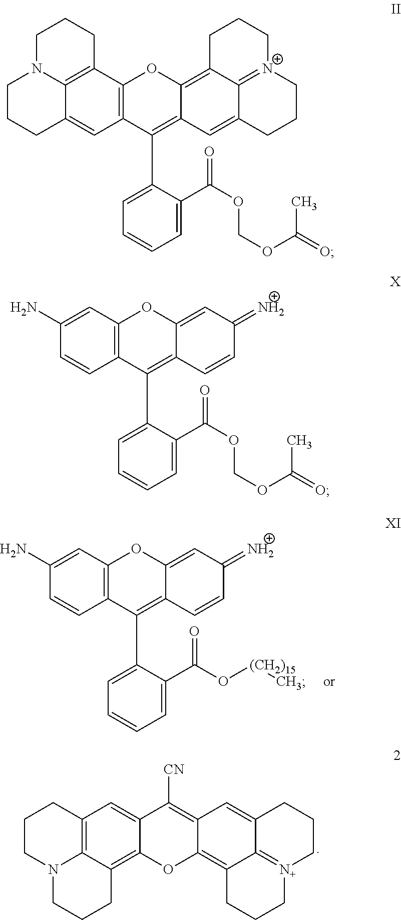

14. A kit for measuring the temperature distribution within a living cell, the kit comprising:
(1) a compound of formula I: 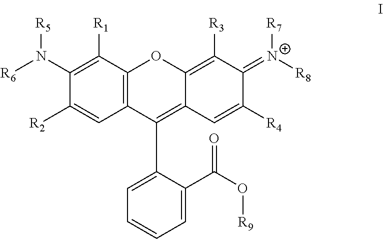
wherein,
R₉ is selected from the group consisting of a hydrocarbyl of 1-22 carbon atoms, an alkyl of 1 to 3 carbon atoms substituted with an ester group of 2-3 carbon atoms and an alkyl of 1 to 3 carbon atoms substituted with an aryl,
R₅, R₆, R₇, R₈ are independently selected from a hydrocarbyl, and all of R₁, R₂, R₃, R₄ are H or a lower hydrocarbyl;
or
R₉ is selected from the group consisting of a hydrocarbyl of 1-22 carbon atoms, an alkyl of 1 to 3 carbon atoms substituted with an ester group of 2-3 carbon atoms and an alkyl of 1 to 3 carbon atoms substituted with an aryl, and
R₅ and R₁, R₆ and R₂, R₇ and R₃, R₈ and R₄ are connected into a six-membered ring;
(2) auxiliary reagents used in cell staining;
(3) containers for accomodating the above compounds and auxiliary reagents; and
(4) instruction manual for using said compound to measure temperature distribution inside a living cell.

15. The kit of claim 14, wherein the compound is the following compound:

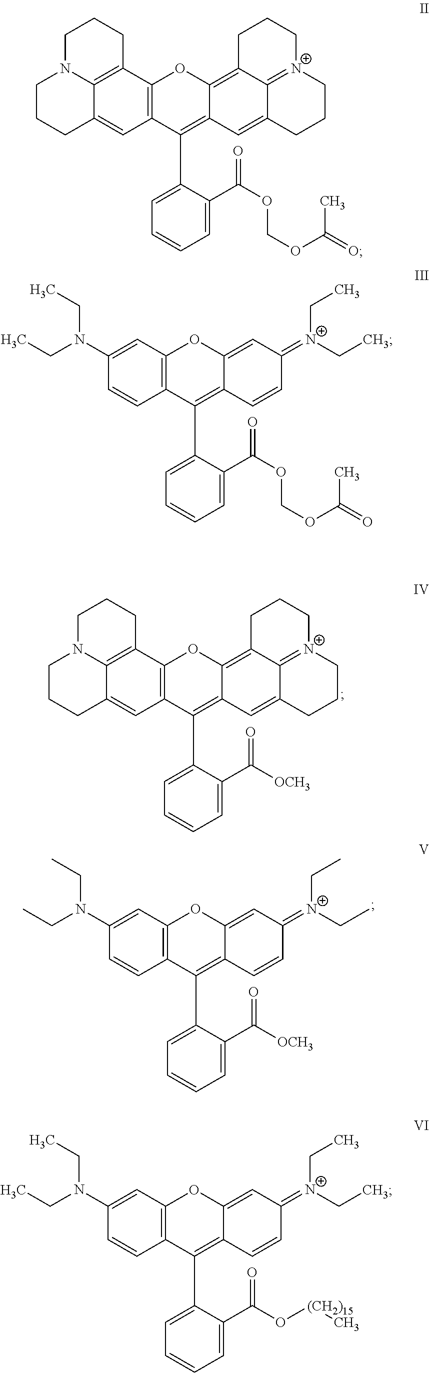

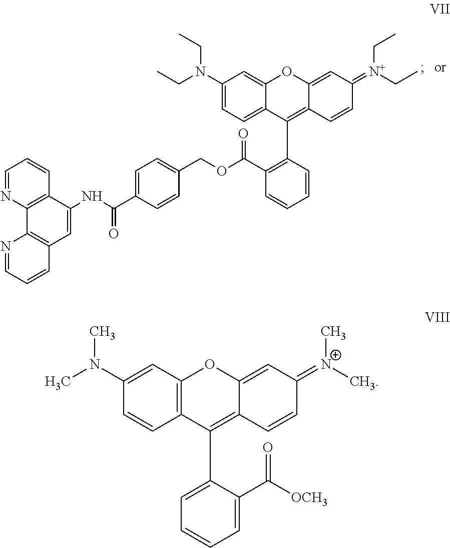

16. The kit of claim 14, wherein the kit also comprises the following compounds: 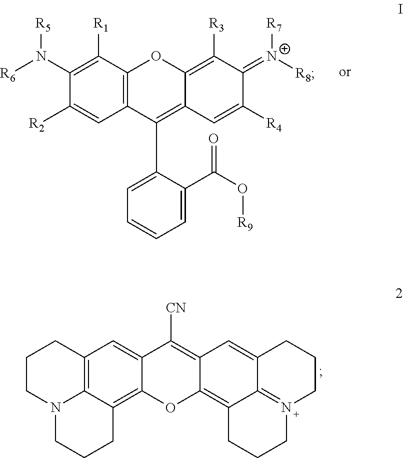
wherein,
R₉ is a hydrocarbyl of 1-22 carbon atoms, or an alkyl of 1 to 3 carbon atoms substituted with an ester group of 2-3 carbon atoms,
all of R₅, R₆, R₇, R₈ are H, and
all of R₁, R₂, R₃, R₄ are H or a lower hydrocarbyl;
or
R₉ is a hydrocarbyl of 1-22 carbon atoms, or an alkyl of 1 to 3 carbon atoms substituted with an ester group of 2-3 carbon atoms, and
R₅ and R₁, R₆ and R₂, R₇ and R₃, R₈ and R₄ are connected into a six-membered ring.

17. The kit of claim 16, wherein the compound is the following compound: [chemical structure]
